(19) United States
(12) Patent Application Publication
(10) Pub. No.: US 2015/0223433 A1
(43) Pub. Date: Aug. 13, 2015

(54) METHOD AND SYSTEM FOR MONITORING ACTIVITY OF AN ANIMAL

(71) Applicant: Agency for Science, Technology and Research, Singapore (SG)

(72) Inventors: Rapeechai Navawongse, Singapore (SG); Deepak Choudhury, Singapore (SG); Adam Claridge-Chang, Sing (SG); Zhiping Wang, Singapore (SG)

(21) Appl. No.: 14/619,649

(22) Filed: Feb. 11, 2015

(30) Foreign Application Priority Data
Feb. 11, 2014 (SG) 201401132-4

(51) Int. Cl. A01K 29/00; G06T 7/00; G01F 23/00; G06K 9/00; A01K 1/03; A01K 5/00

(52) U.S. Cl. CPC A01K 29/005; A01K 1/031; A01K 5/00; G01F 23/0061; G06K 9/00369; G06K 9/00624; G06T 7/004; G06T 2207/10016

(57) ABSTRACT

A method of monitoring activity of an animal, the method comprising the steps of: retaining the animal in an enclosed chamber; controlling a food supply to the animal in the enclosed chamber as a first stimulus; and monitoring the activity of the animal in response to the first stimulus, wherein monitoring the activity of the animal comprises analysing change in a volume of food in the food supply and location of the animal in the enclosed chamber over a predetermined period of time.

### CLAIM OF PRIORITY

This application claims the benefit of priority of Singapore Patent Application Serial No. 201401132-4, filed on Feb. 11, 2014, the benefit of priority of which is claimed hereby, and which is incorporated by reference herein in its entirety.

### TECHNICAL FIELD

The present invention relates broadly to a method and a system for monitoring activity of an animal.

### BACKGROUND

Feeding is a common behaviour among living beings. Most species, from single cell organisms to mammals, would not be able to survive without food. The foraging strategies and choices of food of various species have been studied intensively in research activities. Many molecular, genetic tools and techniques are available for this purpose.

Basic biochemistry and molecular pathways are highly similar between invertebrates and humans, especially in feeding and metabolism regulation system. Thus, various behaviours and disease researches have been carried out on invertebrates. Drosophila, which is a type of fly species, is a widely used invertebrate for such research because it can be easily handled and maintained.

Various Drosophila behaviour assays are available to monitor the basic activities of Drosophila, such as rapid negative geotaxis assay, beam break counter monitoring for sleeping behaviour, etc. However, the molecular and behavioural mechanisms driving the feeding behaviours are still unclear. Further, there is no assay that observes more than one basic activity and behaviour of Drosophila simultaneously.

A need therefore exists to provide a system for monitoring activity of a small animal that seeks to address at least some of the problems above and to provide a useful alternative.

### SUMMARY

According to a first aspect to the present invention, there is provided a method of monitoring activity of an animal, the method comprising the steps of:

retaining the animal in an enclosed chamber;

controlling a food supply to the animal in the enclosed chamber as a first stimulus; and

monitoring the activity of the animal in response to the first stimulus,

wherein monitoring the activity of the animal comprises analysing change in a volume of food in the food supply and location of the animal in the enclosed chamber over a predetermined period of time.

Controlling the food supply to the animal may comprise dispensing the food via a microfluidic channel, the microfluidic channel having predetermined dimensions.

Controlling the food supply may further comprise operating a pump attached to the microfluidic channel.

Analysing the change in the volume of food may comprise measuring height of a fluid containing the food in the microfluidic channel over the predetermined period of time.

Analysing the location of the animal over the predetermined period of time may comprise obtaining video data of the animal using an imaging device and extracting positional information of the animal from the video data.

Extracting positional information may comprise determining a position of centroid of the animal body.

Extracting positional information may comprise determining an orientation of the animal.

Controlling the food supply to the animal may be based on the positional information of the animal.

The method may further comprise the steps of:

providing a second stimulus different from the first stimulus to the animal; and

monitoring the activity of the animal in response to a combination of the first and second stimulus.

The second stimulus may comprise one selected from a group consisting of a light, a sound, an electric current, an odour and a temperature change.

According to a second aspect of the present invention, there is provided a system for monitoring activity of an animal, the system comprising:

an enclosed chamber for retaining the animal;

a food supply for feeding the animal to provide a first stimulus; and

monitoring means configured to monitor the activity of the animal in response to the first stimulus;

wherein the monitoring means is configured to analyse change in a volume of food in the food supply and location of the animal in the enclosed chamber over a predetermined period of time for monitoring the activity of the animal.

The food supply may be configured to dispense the food via a microfluidic channel in fluid communication with the enclosed chamber, the microfluidic channel having predetermined dimensions.

The system may further comprise a pump attached to the microfluidic channel and operable to supply food to the microfluidic channel.

An end of the microfluidic channel adjacent to the enclosed chamber may comprise a feeding region, the feeding region configured for access by the animal.

The monitoring means may be configured to measure a height of a fluid containing the food in the microfluidic channel over the predetermined period of time for analysing the change in the volume of the food.

The monitoring means may be configured to obtain video data of the animal using an imaging device and extract positional information of the animal from the video data for analysing the location of the animal over the predetermined period of time.

The positional information may comprise a position of a centroid of the animal body.

The positional information may comprise an orientation of the animal.

The food supply may be operable based on the positional information of the animal.

The system may further comprise:

stimulating means configured to provide a second stimulus different from the first stimulus to the animal; and

monitoring means configured to monitor the activity of the animal in response to a combination of the first and second stimulus.

The second stimulus may comprise one selected from a group consisting of a light, a sound, an electric current, an odour and a temperature change.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1A shows a perspective clear view of a life support device for an animal according to an example embodiment.

FIG. 1B shows a perspective opaque view of the life support device of FIG. 1A.

FIG. 10 shows a front view of the life support device of FIG. 1A with example linear dimensions.

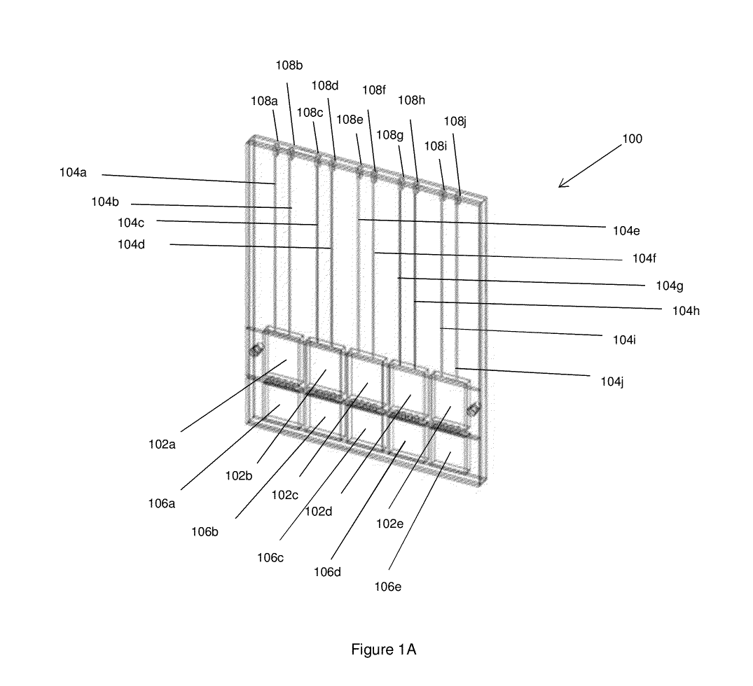

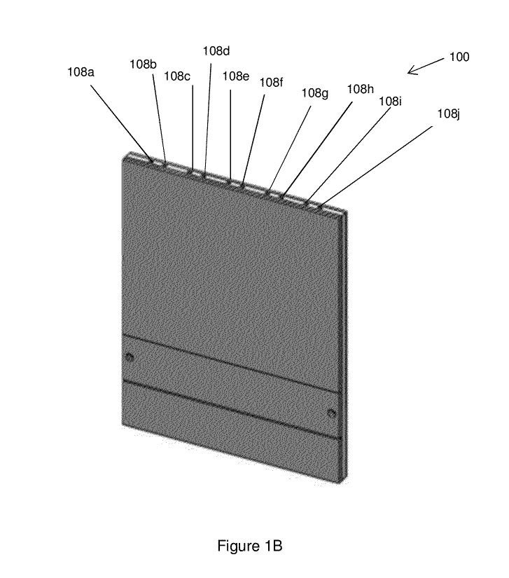

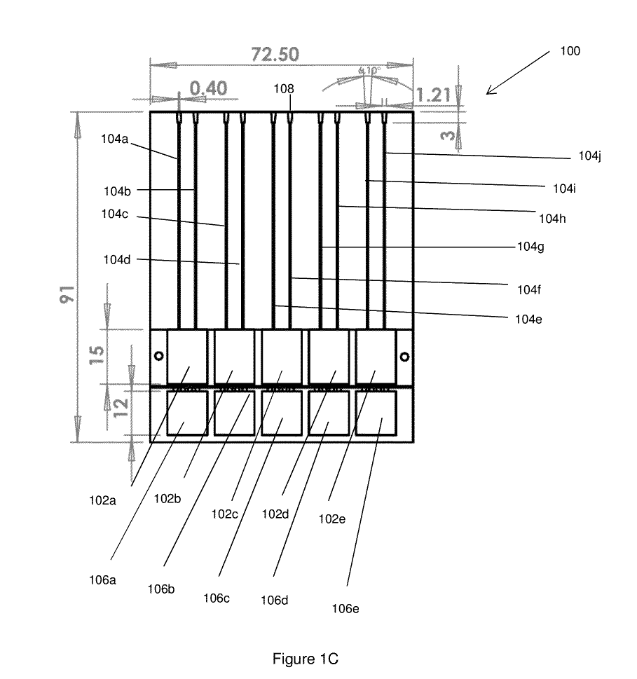

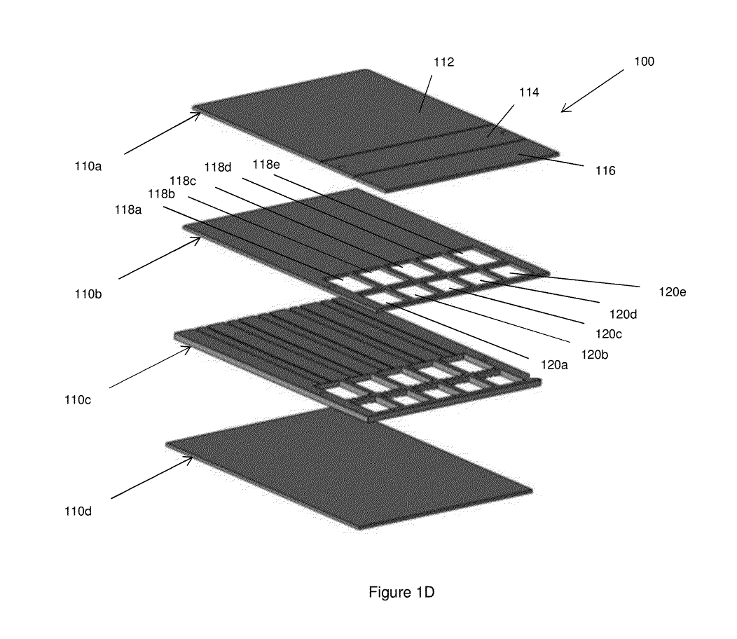

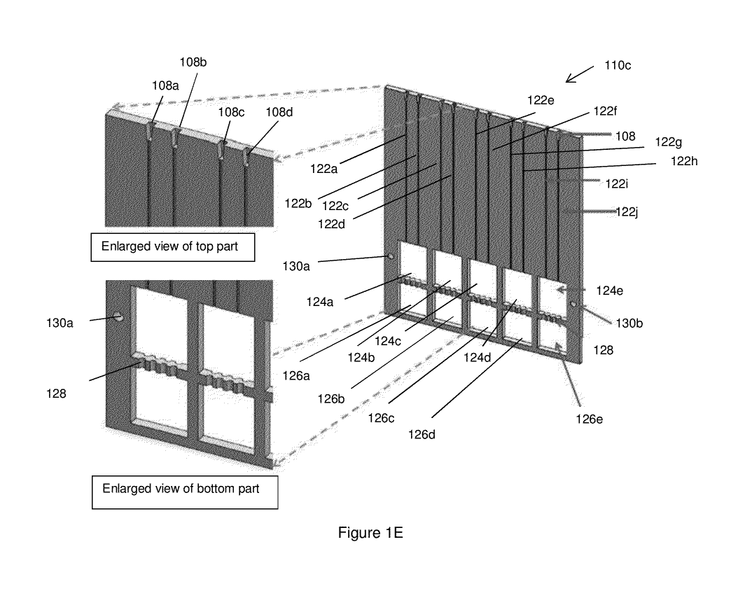

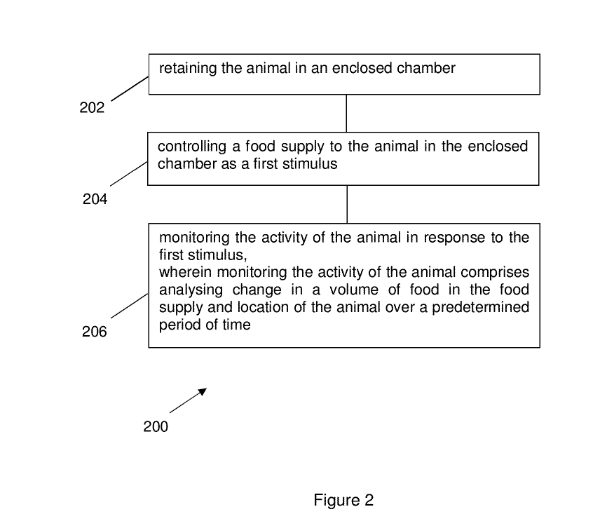

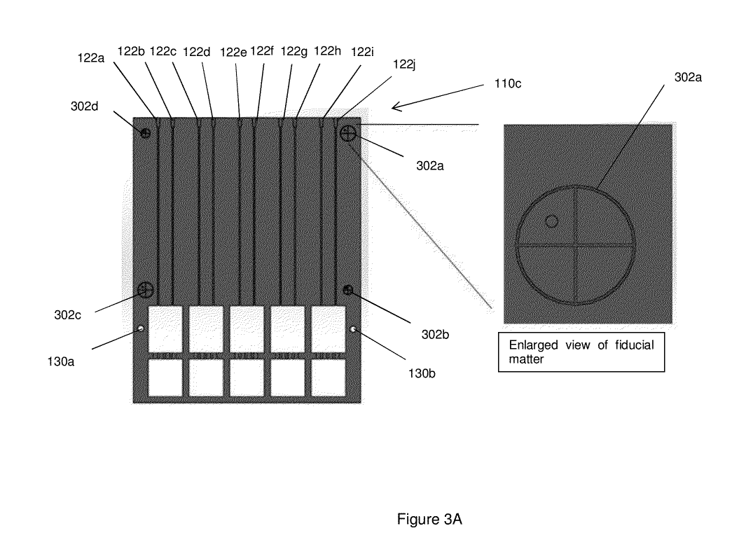

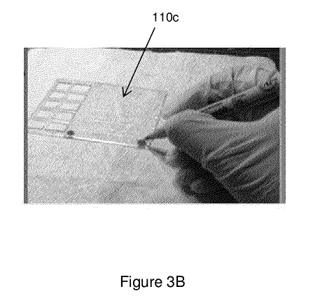

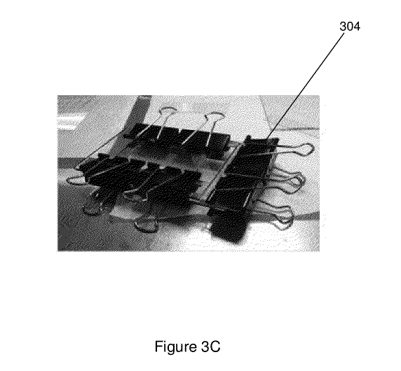

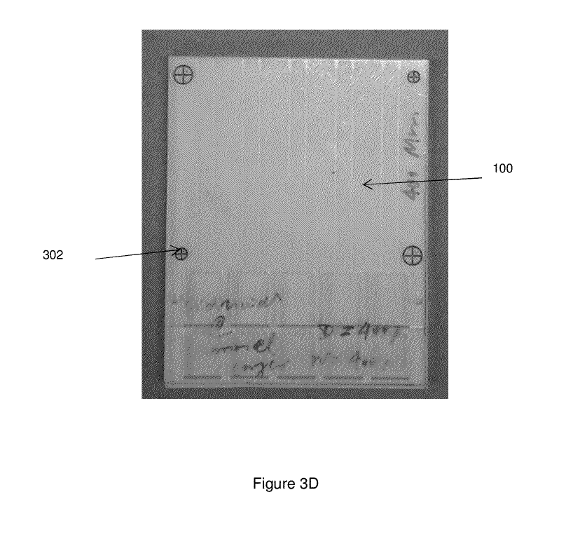

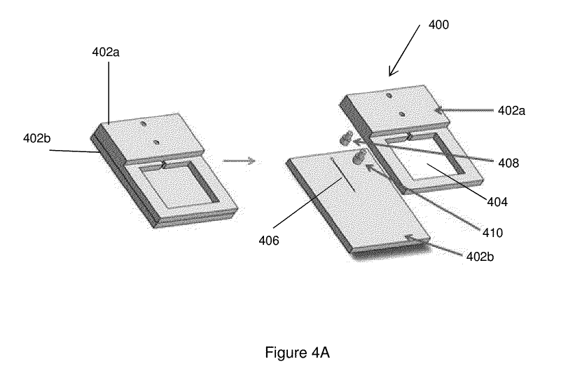

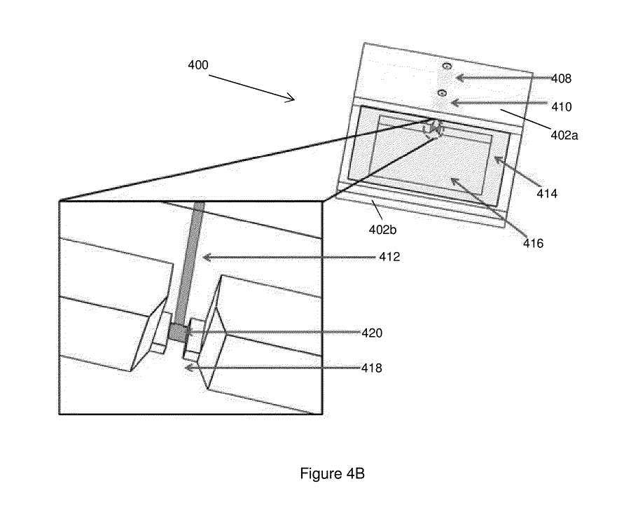

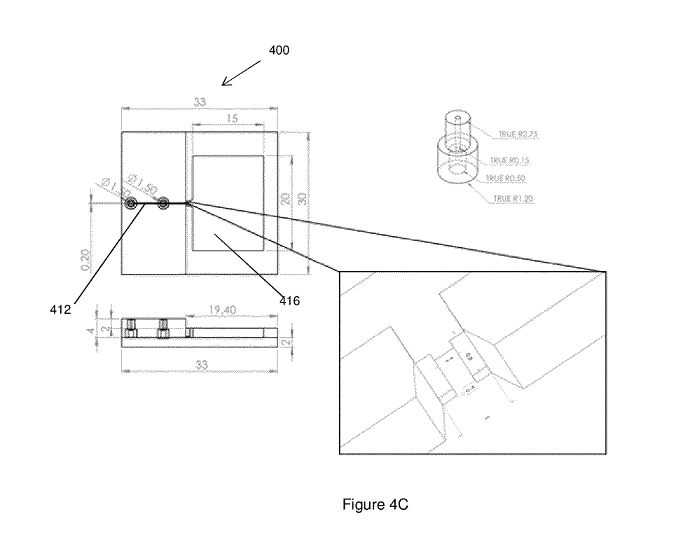

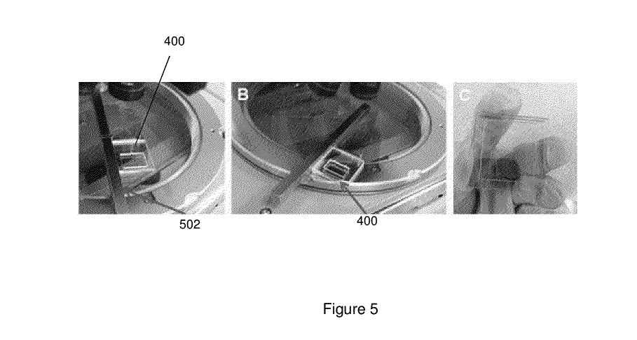

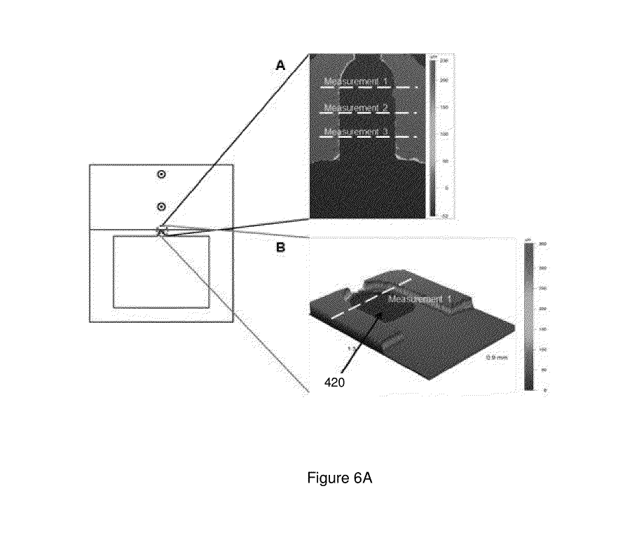

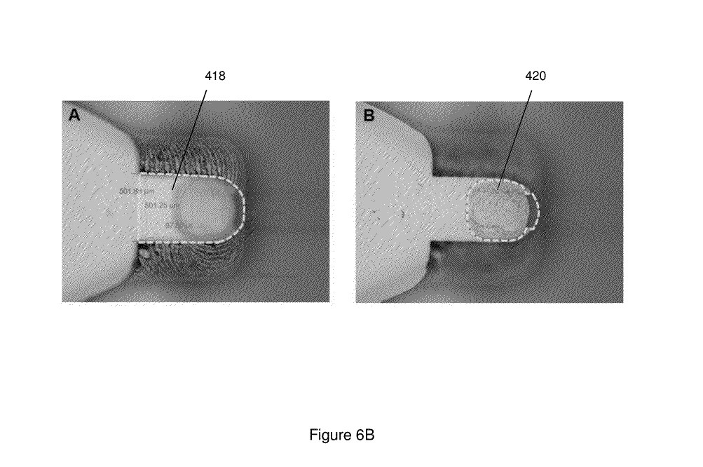

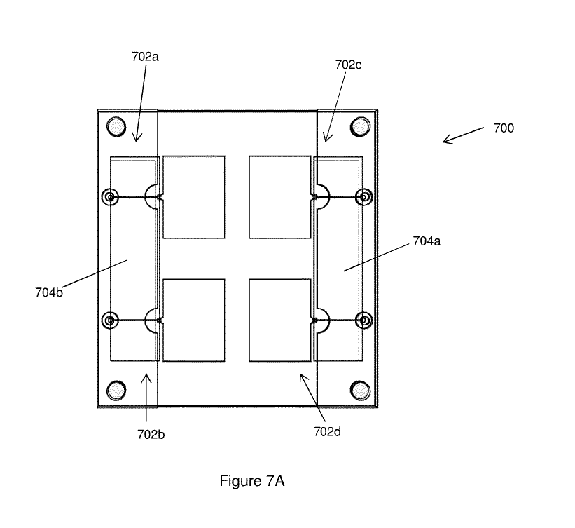

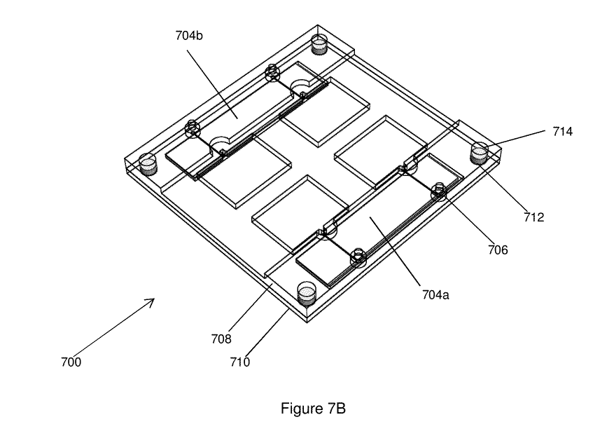

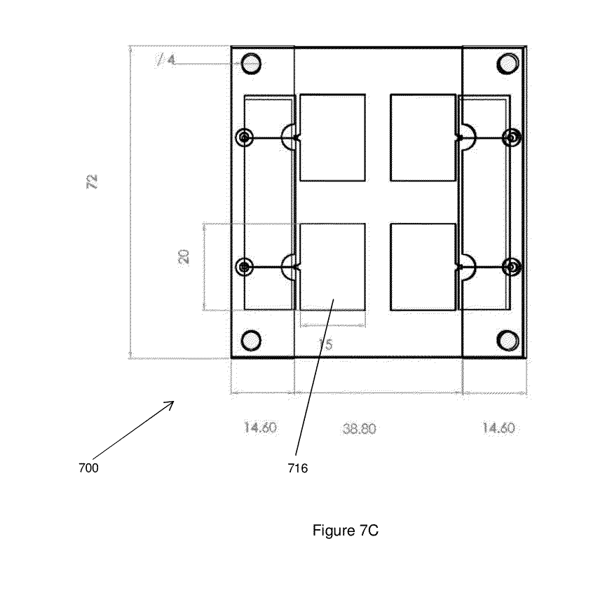

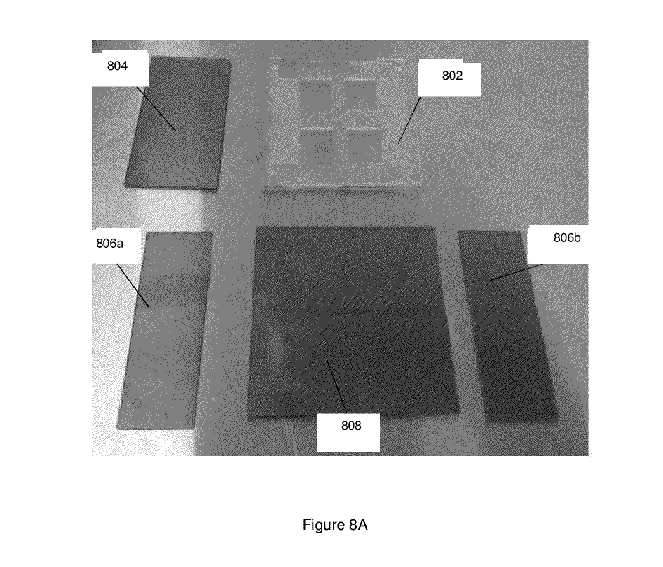

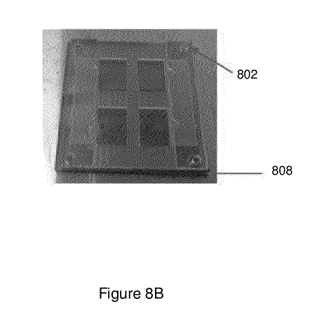

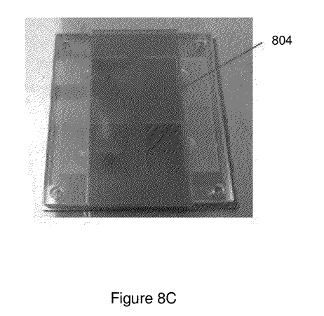

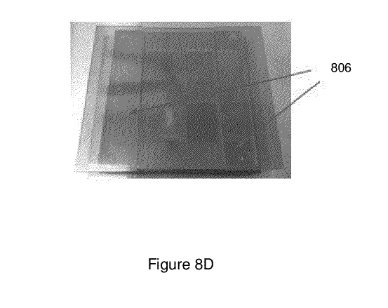

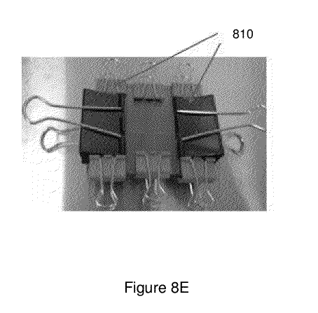

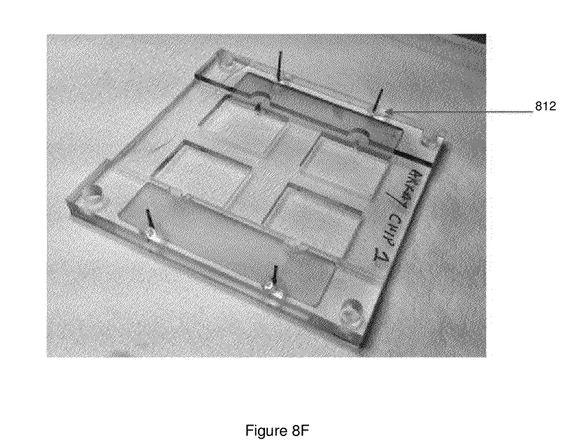

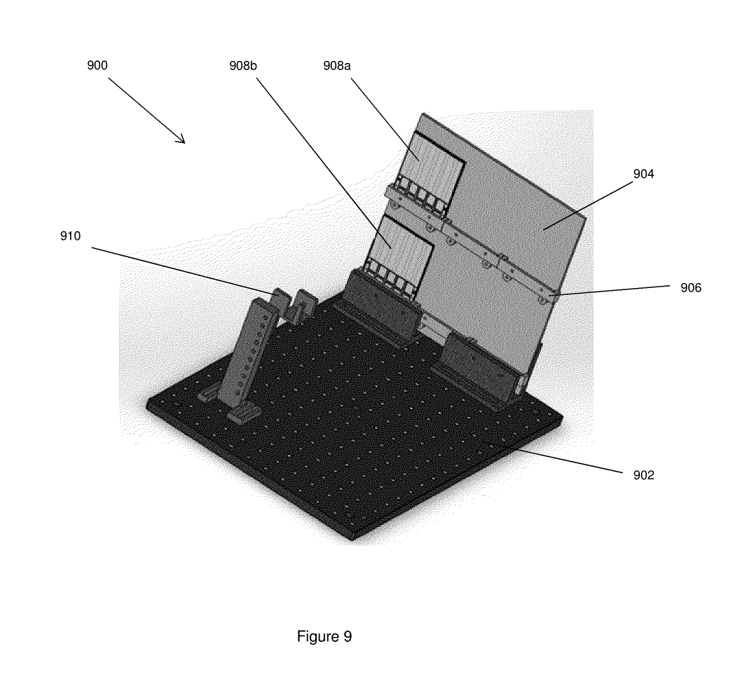

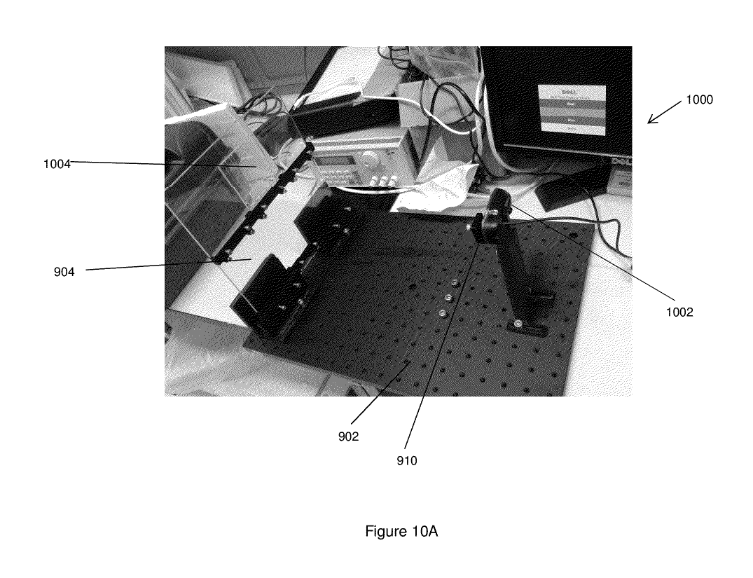

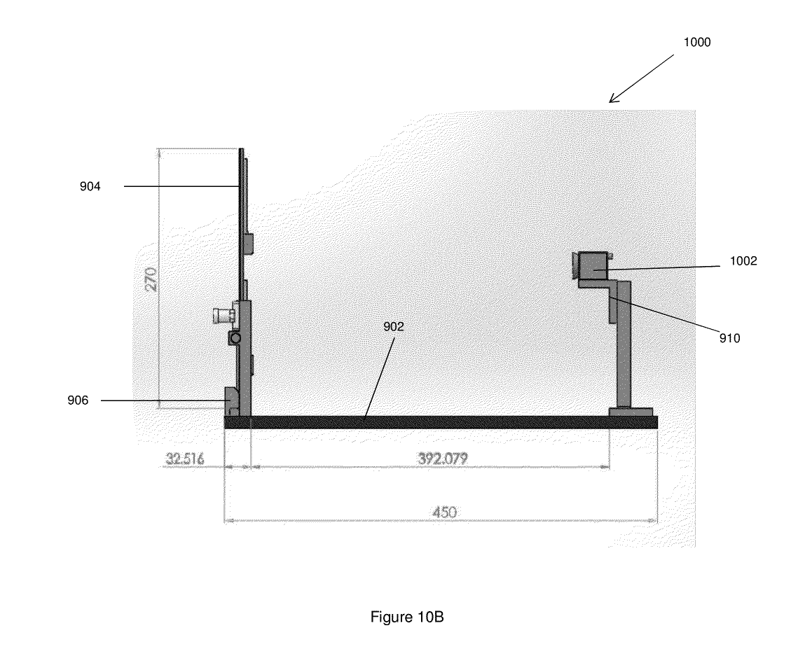

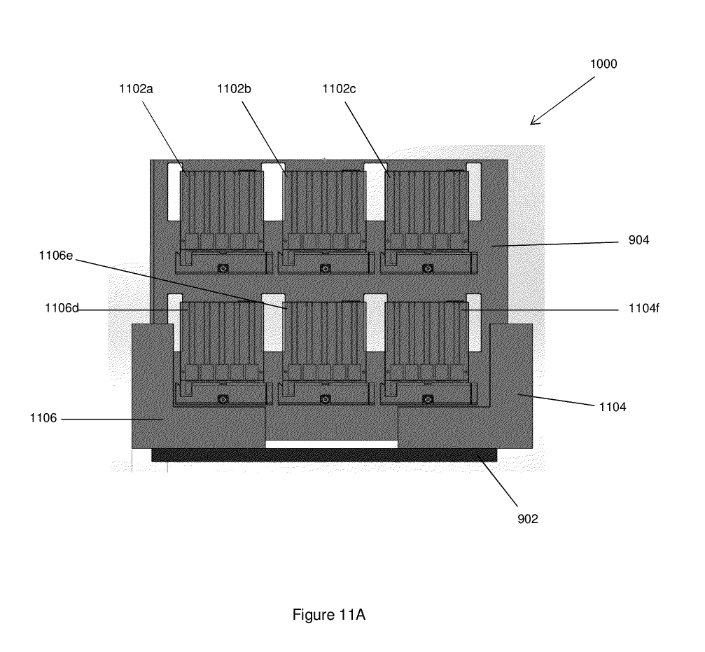

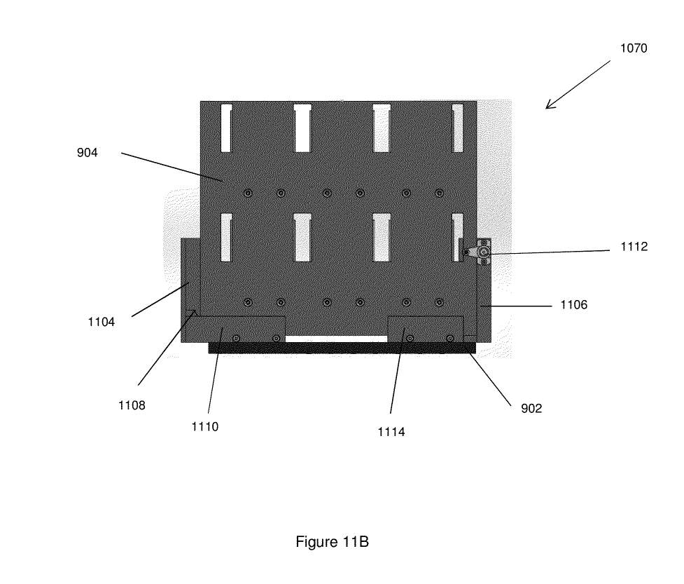

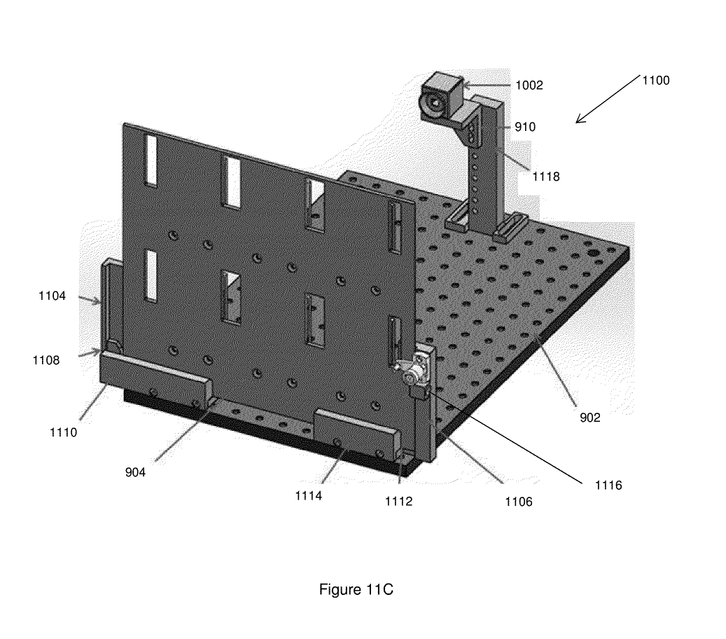

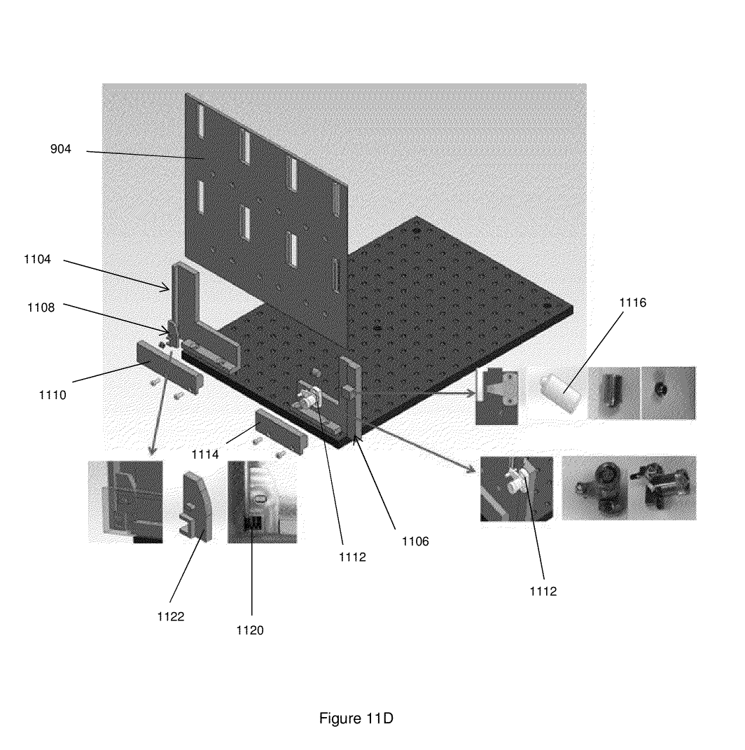

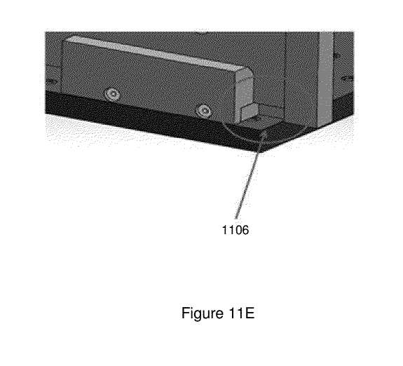

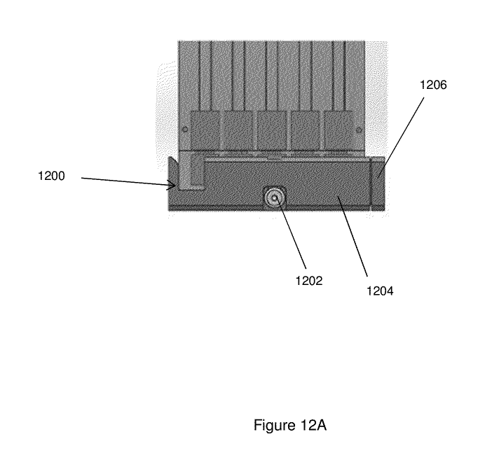

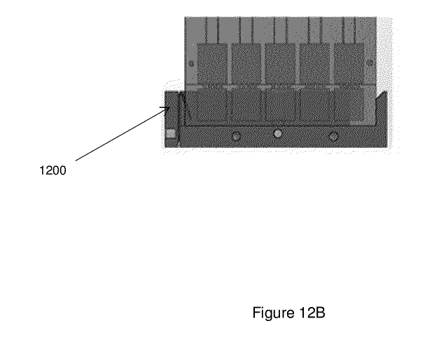

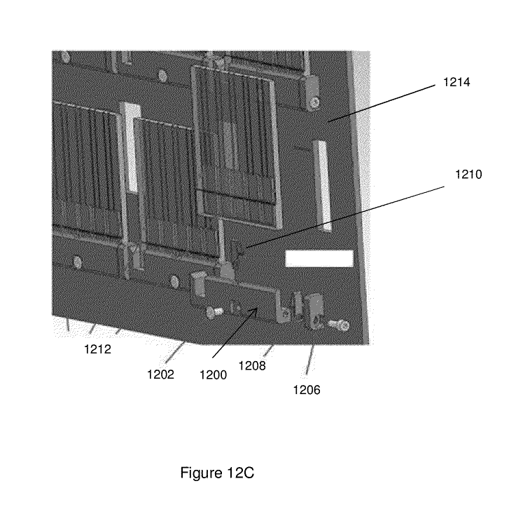

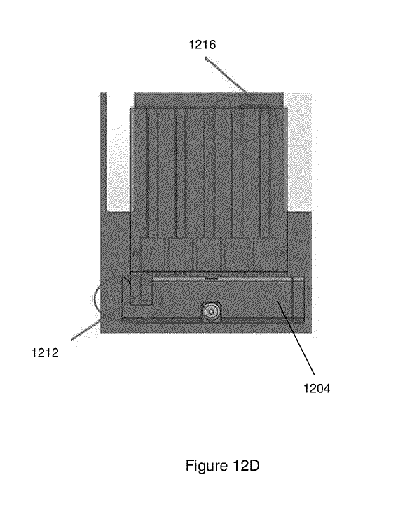

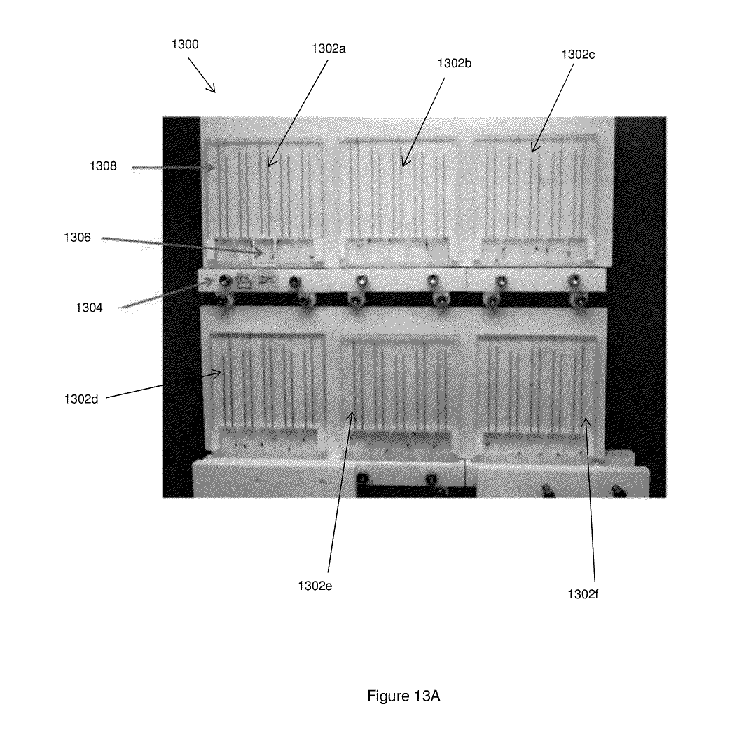

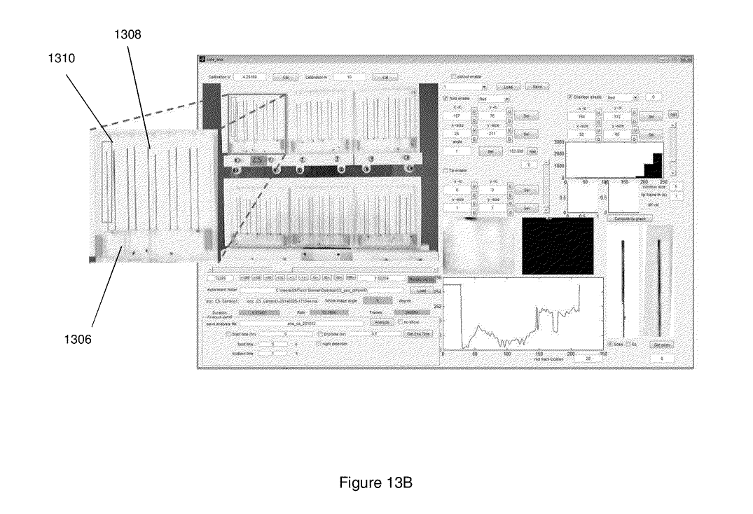

FIG. 1D shows an exploded view of a life support device of FIG. 1A.

FIG. 1E shows a perspective view of the third layer and enlarged views of top and bottom parts of the third layer of FIG. 1D.

FIG. 2 shows a flow chart illustrating a method of monitoring activity of an animal according to an example embodiment.

FIG. 3A shows a front view of the third layer in FIG. 1D with fiducial markers.

FIG. 3B shows an image illustrating the third layer in FIG. 1D with fiducial markers being coloured.

FIG. 3C shows an image illustrating the bonded layers in FIG. 1D undergoing thermal bonding process.

FIG. 3D shows an image illustrating the life support device in FIG. 1D with fiducial markers after thermal bonding process.

FIG. 4A shows a perspective view and an exploded view of a life support device for an animal according to a further example embodiment.

FIG. 4B shows a perspective view of the life support device and an enlarged view of the fluid channel in FIG. 4A.

FIG. 4C shows a front view and side view of the life support device and an enlarged view of the feeding region in FIG. 4A with example linear dimensions.

FIG. 5 shows images illustrating the steps of fabricating the life support device described in FIGS. 4A-4C.

FIG. 6A shows images of the feeding region in FIG. 4B when measured with a 3D optical profiler.

FIG. 6B shows images of the feeding region and paddle shape region in FIG. 4B when measured with ImageJ program.

FIG. 7A shows a front view of a support device for an animal according to an example embodiment.

FIG. 7B shows a perspective view of the life support device in FIG. 7A.

FIG. 7C shows a front view of the life support device in FIGS. 7A and 7B with example linear dimensions.

FIG. 8A shows an image illustrating examples of various items used to fabricate the life support device in FIG. 7A.

FIG. 8B shows an image illustrating the bonded layer in FIG. 8A after being placed on a large glass slide.

FIG. 8C shows an image illustrating the medium glass slide after being placed on a recess on the top centre of the bonded layer in FIG. 8A.

FIG. 8D shows an image illustrating the microscope glass slides after being placed on both sides of the bonded layer in FIG. 8A.

FIG. 8E shows an image illustrating the life support device in FIG. 8A undergoing thermal bonding process.

FIG. 8F shows an image illustrating the step of attaching needles to the inlets of the bonded layer in FIG. 8A.

FIG. 9 shows a back perspective view of a system for observing activity of an animal according to an example embodiment.

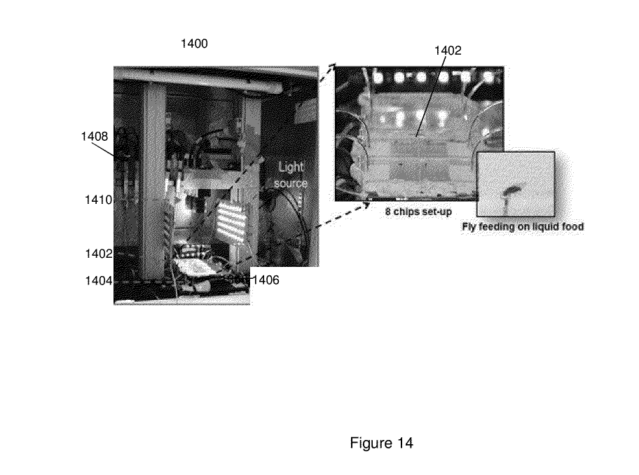

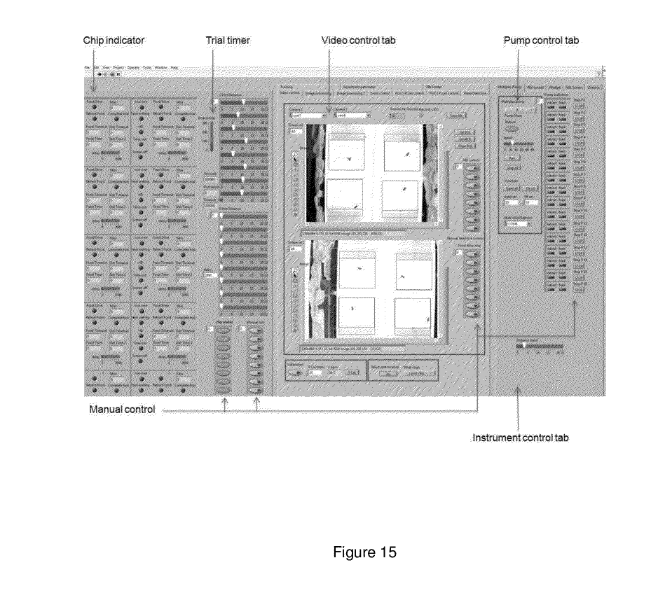

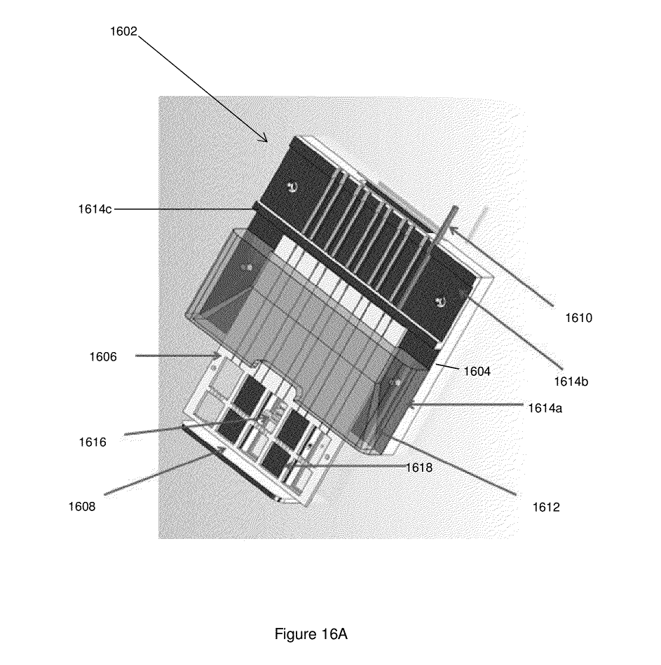

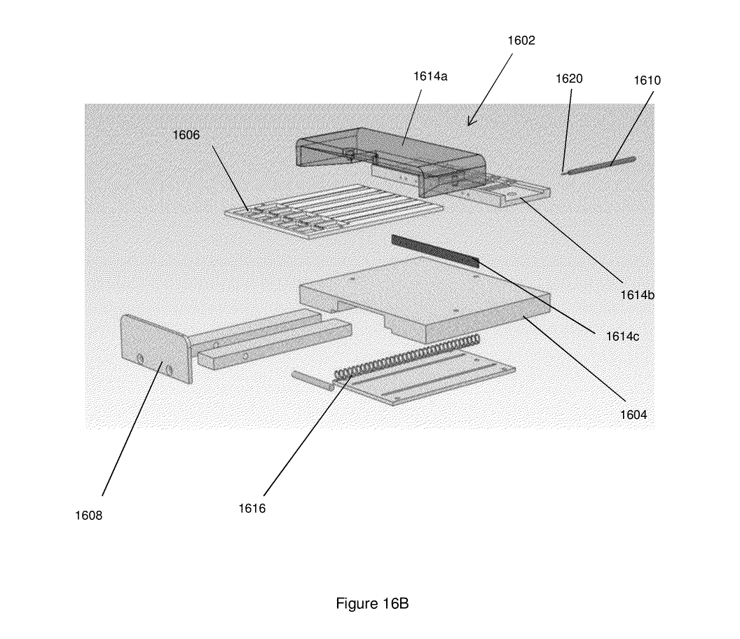

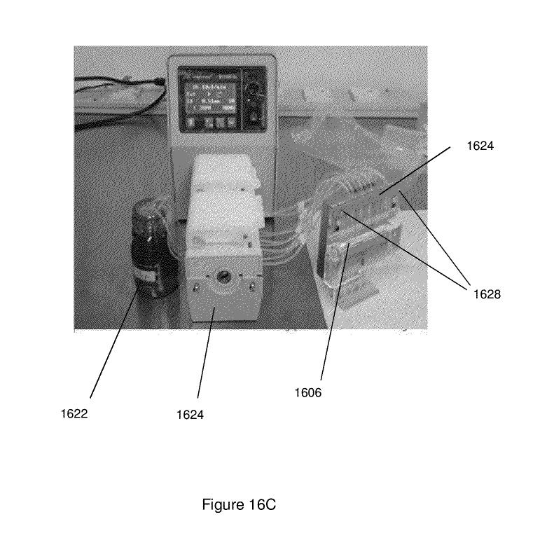

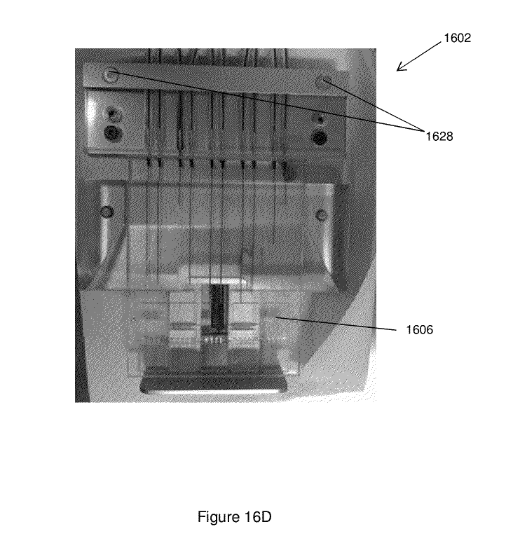

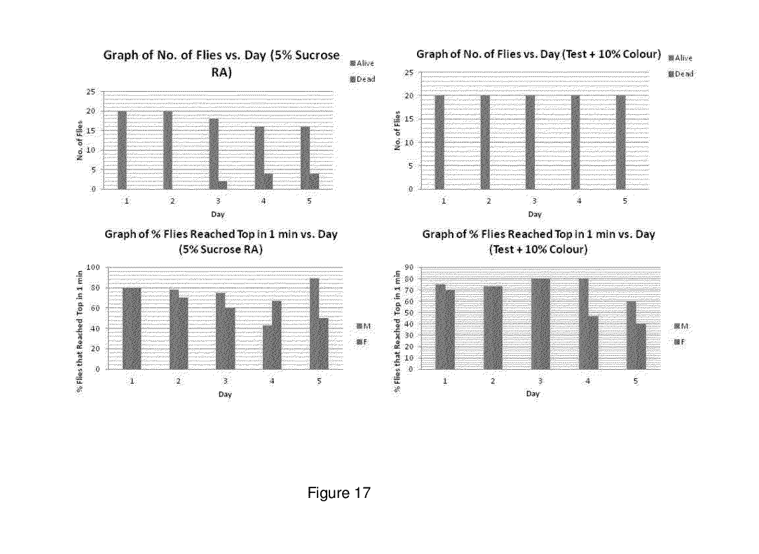

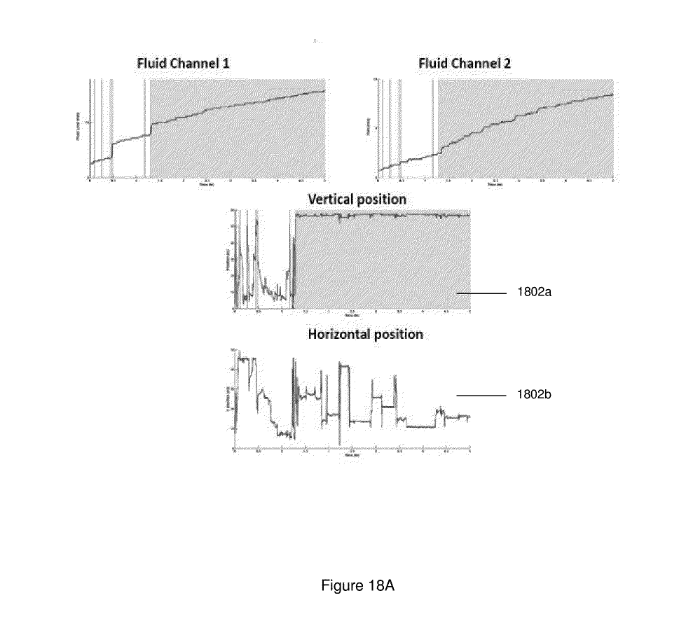

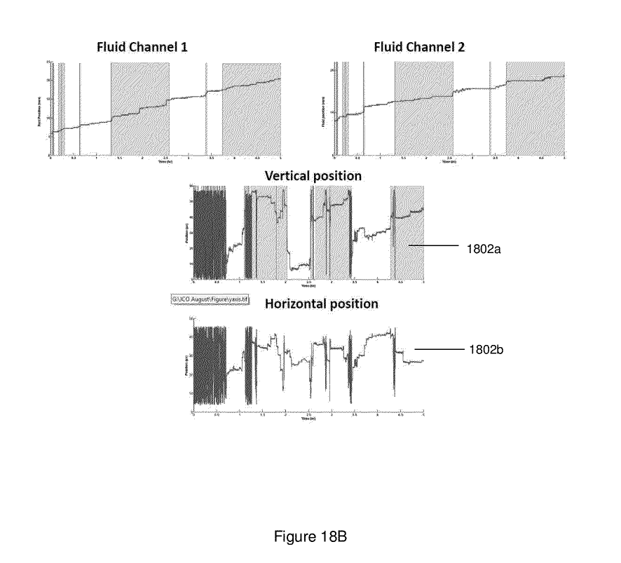

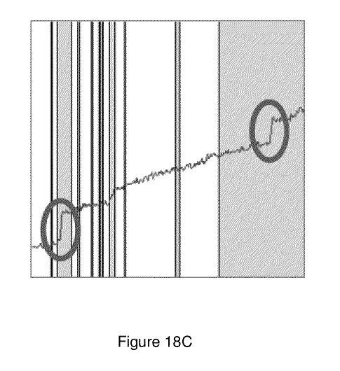

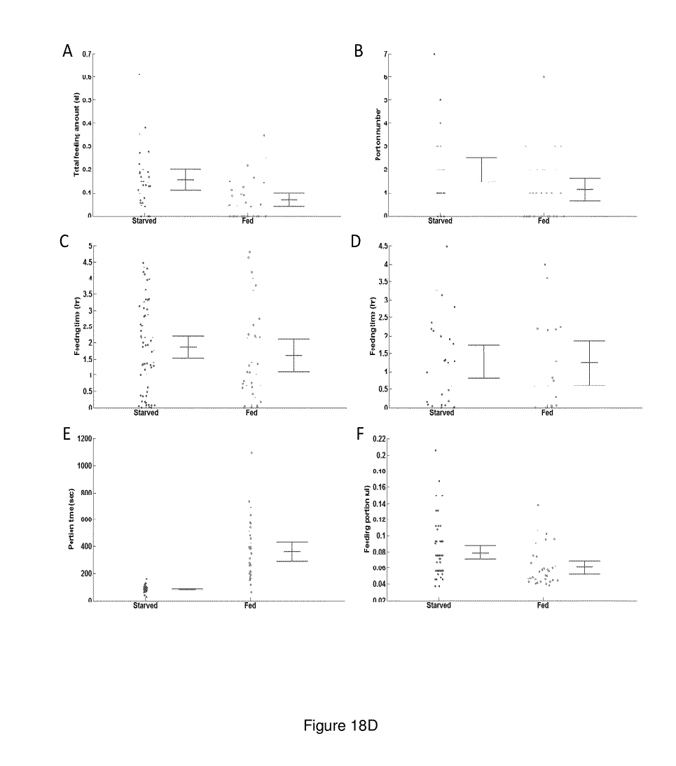

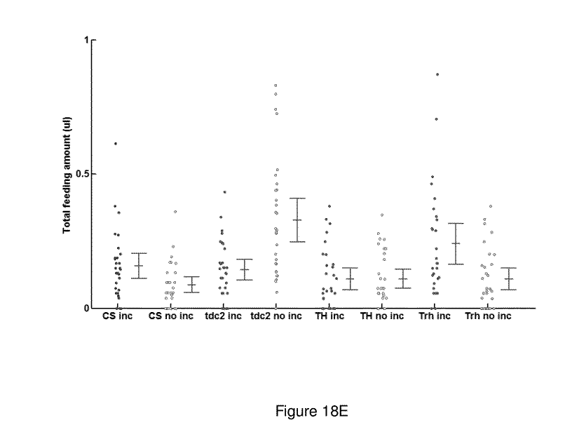

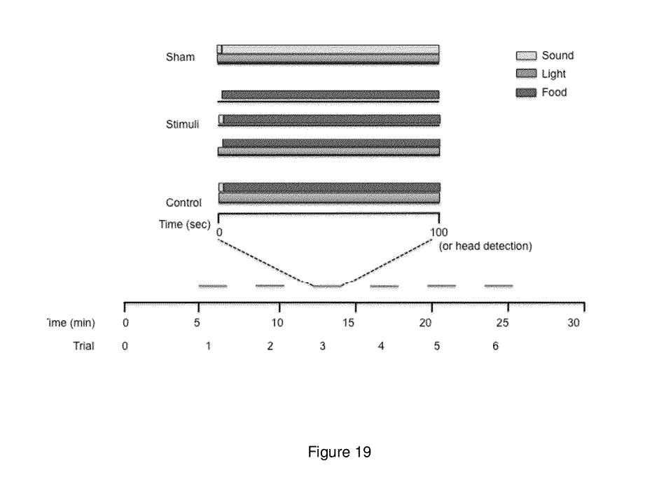

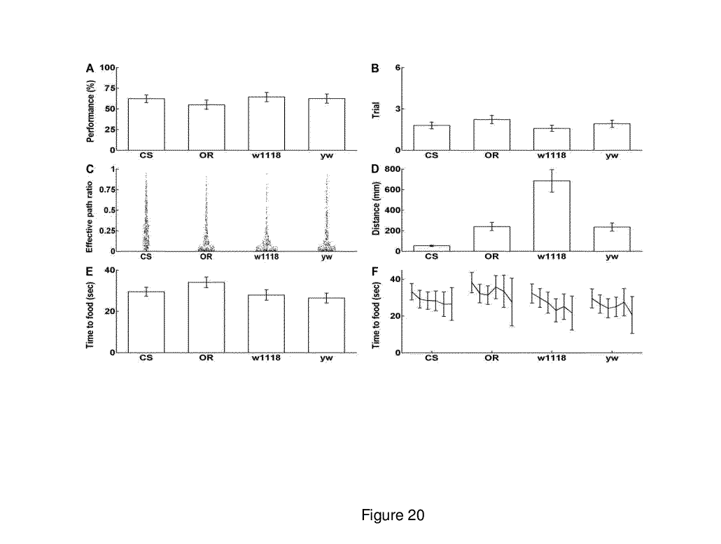

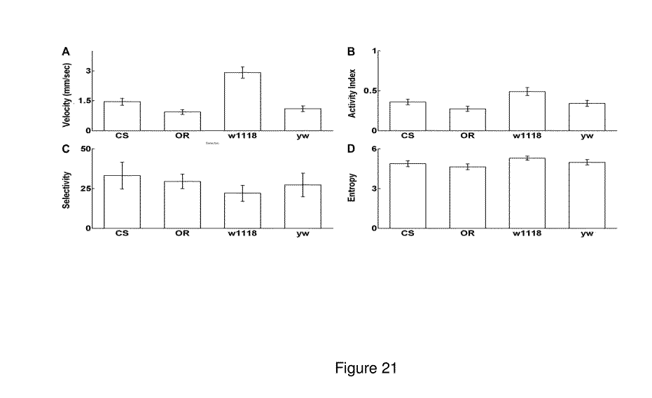

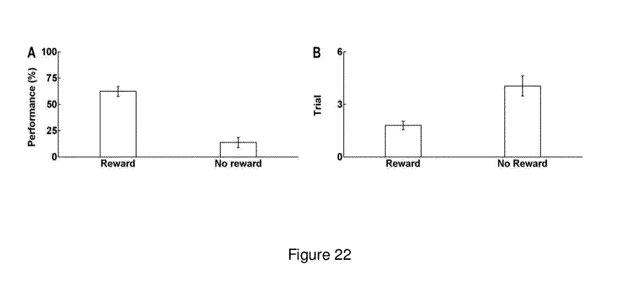

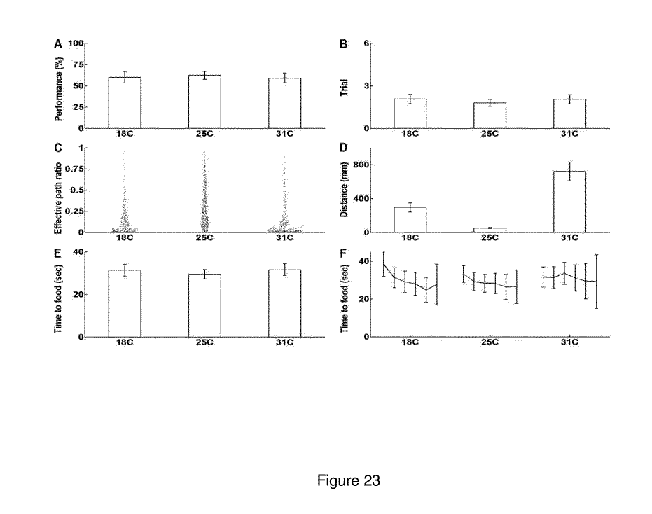

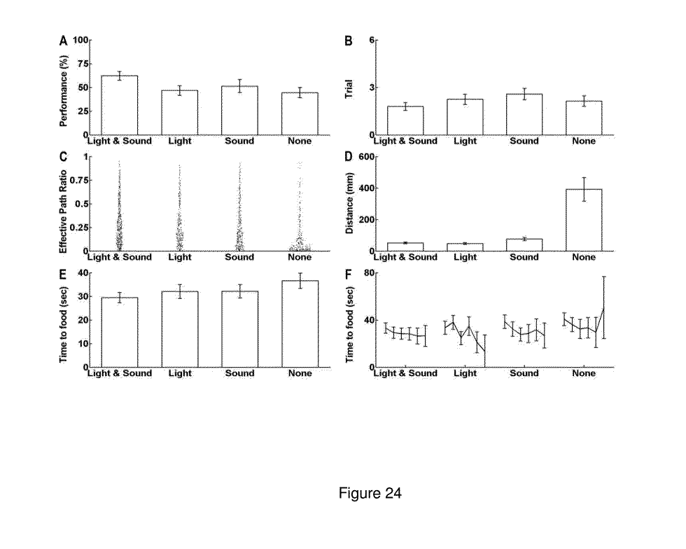

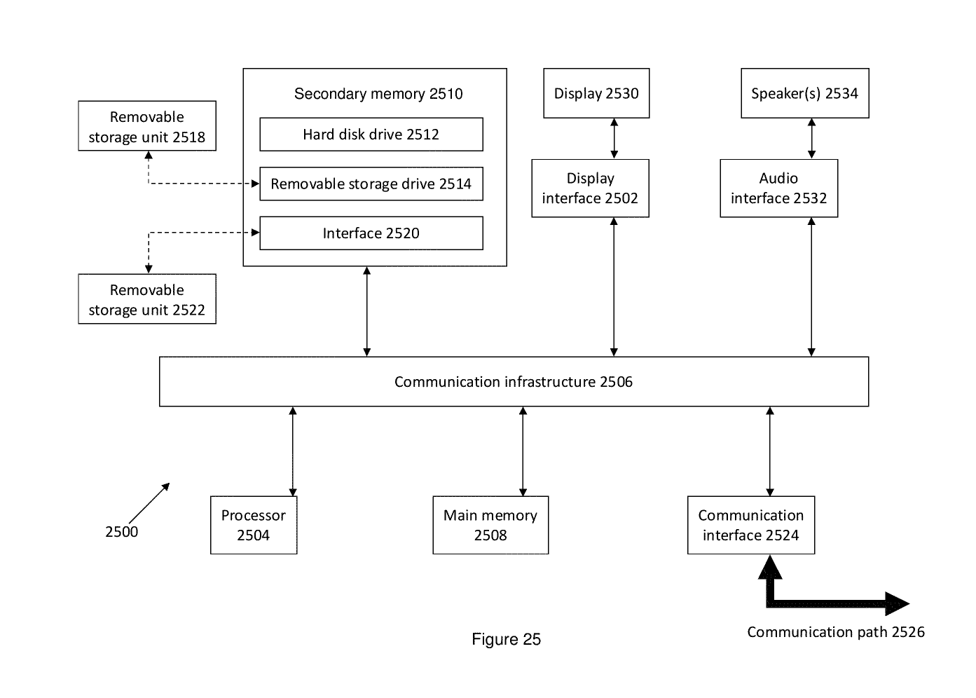

FIG. 10A shows an image illustrating a back perspective view of a system for monitoring activity of an animal according to a further example embodiment.

FIG. 10B shows a right side view of the system of FIG. 10A with example linear dimensions.

FIG. 11A shows a back view of the system in FIG. 10A.

FIG. 11B shows a front view of the system in FIG. 10A.

FIG. 11C shows a front perspective view of the system in FIG. 10A.

FIG. 11D shows an exploded view and configurations of the left side member and right side member in FIGS. 11A-110.

FIG. 11E shows an enlarged view of the right hand member in FIG. 11A-11D after the holder panel is installed.

FIG. 12A shows a back view of a slot according to an example embodiment.

FIG. 12B shows a front view of the slot in FIG. 12A.

FIG. 12C shows an exploded view of the slot in FIG. 12A.

FIG. 12D shows a back view of the slot in FIGS. 12A-12C after the life support device is installed.

FIG. 13A shows an image illustrating a holder panel and six life support devices during an experiment according to an example embodiment.

FIG. 13B shows an image illustrating an interface of an example computer program suitable for use in an example embodiment.

FIG. 14 shows an image illustrating a system for monitoring activity of an animal and an enlarged view of the life support device according to an example embodiment.

FIG. 15 shows an image illustrating an interface of an example computer program used to control the system in FIG. 14.

FIG. 16A shows a perspective view of a refilling device according to an example embodiment.

FIG. 16B shows an exploded view of the refilling device in FIG. 16A.

FIG. 16C shows an image illustrating the arrangement of various items used to refill the life support device in FIG. 16A.

FIG. 16D shows an image illustrating the refilling device in FIG. 16A when used to refill the life support device.

FIG. 17 shows graphs illustrating survival rate and climbing assay results of the experiment according to an example embodiment.

FIG. 18A shows graphs illustrating the results of an experiment including the fluid measurements of channels and locations of flies over time according to an example embodiment.

FIG. 18B shows graphs illustrating the results of an experiment including the fluid measurements of channels and locations of flies over time according to a further example embodiment.

FIG. 18C shows an enlarged view of a graph illustrating the fluid measurements of a channel according to an example embodiment.

FIG. 18D shows graphs illustrating results of the experiment comparing feeding behaviour of starved and non-starved subjects according to an example embodiment.

FIG. 18E shows a graph illustrating the results of the experiment comparing influence of candidate neurotransmitters on feeding behaviour of flies according to an example embodiment.

FIG. 19 shows a chart illustrating the experiment trials that are carried out according to an example embodiment.

FIG. 20 shows graphs illustrating experimental results conducted on four wild types flies according to an example embodiment.

FIG. 21 shows graphs illustrating analysis results for four wild types flies in a ten minutes pre-trial period according to an example embodiment.

FIG. 22 shows graphs comparing experimental results for a default experiment and a sham experiment according to an example embodiment.

FIG. 23 shows graphs comparing experimental results conducted for three different temperatures settings according to an example embodiment.

FIG. 24 shows graphs comparing experimental results conducted for four different stimuli conditions according to an example embodiment.

FIG. 25 depicts an exemplary computing device.

### DETAILED DESCRIPTION

FIGS. 1A and 1B show perspective clear view and perspective opaque view respectively of a life support device 100 for an animal according to an example embodiment. The life support device 100 comprises at least one chamber 102a-e. The at least one chamber 102a-e is used to retain one or more animal, e.g. insects. The life support device 100 further comprises at least one fluid channel 104a-j, wherein the at least one fluid channel 104a-j is in fluid communication with the chambers 102a-e.

The life support device 100 further comprises at least one humidity cavity 106a-e. The humidity cavities 106a-e are also in fluid communication with the respective chambers 102a-e, to keep the chambers 102a-e at a suitable humidity. As shown in FIG. 1A, each of the chambers 102a-e is in fluid communication with two fluid channels 104a-e and one humidity cavity 106a-e. For example, the chamber 102a in FIG. 1A is connected to fluid channels 104a, 104b and humidity cavity 106a.

During experiment, the fluid channels 104a-j are filled with fluid for feeding the animal. For example, the fluid channels 104a-j are filled with fly media when flies are retained in the chambers 102a-e. The fluid channels 104a-j can also be used to retain water. The channels 104a-j are configured to retain the fluid with capillary force. In other words, the channels 104a-j should be of suitable cross-sectional area such that the fluid will not leak from the fluid channels 104a-j into the chambers 102a-e when the life support device 100 is in upright position. In an embodiment, the cross-sectional of the fluid channels 104a-j is rectangular and the dimensions are 200 μm depth and 400 μm width. For example, the fluid channels can retain a volume of about 4 μl, which is enough to feed a single fly for about 1 to 2 days. It will be appreciated that the above-described fluid channels 104a-j is one of the examples and the fluid channels 104a-j may be of different cross-sectional shapes and cross-sectional area. For example, the width and depth of the fluid channel 104a-j can be in the range of about 1 μm to about 1000 μm.

Each of the channels 104a-j is also in fluid communication with a refill inlet 108a-j at a distal end from the chambers 102a-e. Fluid can be pipetted into the fluid channels 104a-j with a precise volume. In an embodiment, the refill inlet 108a-j is configured to tightly fit a 200 μm pipette tip head. In alternate embodiments, instead of using pipette, the channels 104a-j may also be refilled with a refilling device, as described in detail below with respect to FIGS. 16A-16C. The life support device 100 comprises a plurality of layers (not shown), as described in detail below with respect to FIG. 1D.

FIG. 1C shows front view of the life support device 100 of FIG. 1A with example linear dimensions. The dimensions in FIG. 10 are indicated in the mm unit. As shown in FIG. 10, the dimensions of the life support device 100 are 91 mm height and 72.5 mm width. The heights of the chambers 102a-e and humidity cavity 106a-e are 15 mm and 12 mm respectively. As described above, the width of the fluid channels 104a-j is 0.4 mm (400 μm). The dimensions of the refilling fillets 108a-j are 3 mm height and 1.21 mm width. In the embodiment, the refilling fillets 108a-j are tapered channels, expanding with an angle of about 6 to 10° from the fluid channels 104a-j. It will be appreciated by a person skilled in the art that the life support device 100 may comprise different dimensions and the above-described dimensions are only one of the examples.

FIG. 1D shows an exploded view of the life support device 100 of FIG. 1A. As shown in FIG. 1D, the life support device 100 comprises a plurality of layers, represented in FIG. 1D as 110a-d. The layers 110a-d are stacked on each other and thermally bonded, forming the life support device 100. The process of thermal bonding is described in detail below with respect to FIG. 3C. In the embodiment, a first layer 110a comprises 3 parts. The first part 112 covers the fluid channels 104a-j. The second part is a cover detachable from the rest of the life support device 100. The cover is represented in FIG. 1D as a chamber cover 114 and may be manually fastened to the life support device 100 with one or more fastening member (not shown), for e.g. M2 screws. In an experiment, the chamber cover 114 is attached to the life support device 100 after the flies are placed inside the chambers 102a-e. The third part is a humidity cavity cover 116, forming the humidity cavities 106a-e with other layers 110b-d. The first part 112 and third part 116 are thermally bonded to a second layer 110b.

Further, FIG. 1D shows that the second layer 110b comprises at least one aperture, represented as apertures 118a-e and 120a-e. The second layer 110b is thermally bonded to a third layer 110c. The third layer 110c also comprises the corresponding apertures as the second layer 110b, as described in detail below with respect to FIG. 1E. In addition, a fourth layer 110d is thermally bonded to third layer 110c to complete the assembly of the life support device. It should be noted that the life support device 100, including the detachable cover 114 and the fluid channels 104a-j, is fabricated using transparent material. In an embodiment, the life support device 100 is fabricated using transparent thermoplastic cast acrylic material, for e.g. PMMA-Poly(methyl methacrylate). The fluid in the fluid channels 104a-j and flies in the chambers 102a-e should be clearly visible through the life support device 100. Computer numerical control (CNC) milling machine, injection moulding machine or embossing machine can be used to fabricate the layers 110a-d. It will be appreciated that other transparent materials, e.g. glass, may be used to fabricate the life support device 100.

FIG. 1E shows a perspective view of the third layer 110c and enlarged views of top and bottom parts of the third layer 110c of FIG. 1D. This third layer 110c comprises at least one groove, represented as 122a-j in FIG. 1E. The grooves 122a-j form the fluid channels 104a-j when bonded to second layer 110b. Thus, the dimensions of the groove form the dimensions of the fluid channels 104a-j, i.e. 200 μm depth and 400 μm width.

The third layer 110c also comprises apertures 124a-e and 126a-e, corresponding to apertures 118a-e and 120a-e respectively of the second layer 110b. The apertures 118a-e and 124a-e form the chambers 102a-e; the apertures 120a-e and 126a-e form the humidity cavities 106a-e in the assembled life support device 100. As can be seen in the enlarged view of the bottom part of the third layer 110c in FIG. 1E, apertures 124a-e are connected to apertures 126a-e via 8 hemispherical holes. It is also illustrated that the third layer 110c comprises assembly holes 130a, 130b with internal threads (not shown) to receive the screws (not shown) from the first layer 110a for fastening the chamber cover 114. The enlarged view of the top part of the third layer 110c illustrates the refill inlets 108a-d. Here, it is clearly illustrated that the refill inlet has a cross-sectional area larger than that of the fluid channel 104a-j, as the refill inlet is configured to fit a pipette tip head or other refilling means.

FIG. 2 shows a flow chart 200 illustrating a method of monitoring activity of an animal according to an example embodiment. At step 202, the animal is retained in an enclosed chamber. At step 204, a food supply to the animal in the enclosed chamber is controlled. The controlling of the food supply is a first stimulus. At step 206, the activity of the animal in response to the first stimulus is monitored. Monitoring the activity of the animal comprises analysing change in a volume of food in the food supply and location of the animal in the enclosed chamber over a predetermined period of time.

Some portions of the description which follows are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilizing terms such as “scanning”, “calculating”, “determining”, “replacing”, “generating”, “initializing”, “outputting”, or the like, refer to the action and processes of a computer system, or similar electronic device, that manipulates and transforms data represented as physical quantities within the computer system into other data similarly represented as physical quantities within the computer system or other information storage, transmission or display devices.

The present specification also discloses apparatus for performing the operations of the methods. Such apparatus may be specially constructed for the required purposes, or may comprise a general purpose computer or other device selectively activated or reconfigured by a computer program stored in the computer. The algorithms and displays presented herein are not inherently related to any particular computer or other apparatus. Various general purpose machines may be used with programs in accordance with the teachings herein. Alternatively, the construction of more specialized apparatus to perform the required method steps may be appropriate. The structure of a conventional general purpose computer will appear from the description below.

In addition, the present specification also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the invention.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a general purpose computer. The computer readable medium may also include a hard-wired medium such as exemplified in the Internet system, or wireless medium such as exemplified in the GSM mobile telephone system. The computer program when loaded and executed on such a general-purpose computer effectively results in an apparatus that implements the steps of the preferred method.

FIG. 3A shows a front view of the third layer 110c in FIG. 1D with fiducial markers. In FIG. 3A, fiducial markers 302a-d are engraved at the sides on the third layer 110c adjacent the grooves 122a-j. As illustrated in the enlarged view of the fiducial marker 302a in FIG. 3A, the fiducial markers 302a-d are designed with a cross-hair with reticle. This allows the fiducial markers 302a-d to be used to align the life support device 100 during an experiment, such that the layout information of the life support device 100, e.g. the dimensions of the fluid channels 104a-j, can be determined using an analysis software. Specifically, the alignment functionality may be provided by the addition of a ‘chip setup’ plugin in the analysis software. A ‘chip layout file’, which can be created using chip template setup graphical user interface (GUI), can be selected and loaded into the alignment configuration. The ‘chip layout file’ stores the location of all regions of interest (ROI), including the fiducial markers 302, tracking regions and custom regions of interest (ROI) such as feed tubes and feed ports, as well as embedded images of the chip and the fiducial markers 302 for reference. It will be appreciated that the fiducial markers 302 can also be engraved in other layers of the life support device 100.

FIGS. 3B-3D shows images illustrating the steps of incorporating fiducial markers on the third layer 110c of FIG. 1D. As illustrated in FIG. 3B, after fiducial markers 302a-d are engraved on the third layer 110c, the fiducial makers 302a-d are coloured, so that the fiducial markers 302a-d are more clearly visible. In FIG. 3C, the third layer 110c is thermally bonded with others layers 110a, 110b, 110d. The layers 110a-d, other than the chamber cover 114, are placed in a borosilicate glass pieces of about 4 mm thick (not shown) and the set-up is tightened with binder clips 304. The assembly is then placed in a hot air oven at 125° C. for 1 hour. The bonded layers 110a-d are then cooled for about 1 hour. After thawing the assembly, tapping was done for the two assembly holes 130a, 130b at the life support device 100. FIG. 3D illustrates a completed life support device 100 with fiducial markers 302a-d clearly visible. The life support device 100 is then tested for any leaks by dispensing coloured food dye through all the fluid channels 104a-j. The chamber cover 114 can be fastened to the thermally bonded layers with screws (not shown) after the insects are placed into the chambers 102a-e. It will be appreciated by a person skilled in the art that the layers 110a-d can be bonded by other methods, e.g. laser bonding, ultrasonic bonding, adhesive bonding etc.

Identification means in the form of a one-dimensional bar code and/or a two-dimensional data matrix code may be pasted on the sides or on one of the humidity cavities of the life support device 100. The bar code and data matrix code can be configured to include data corresponding to attributes of the life supporting device 100, e.g. model number and a unique serial number of the life support device 100. In an implementation, the bar code may adopt UCC/EAN-128 as the standard and the data matrix may be used to include data of a life support device prototype. The identification means may also include an alphanumeric code. For example in the code ECV2-L7-DC-20140930, the portion “ECV” represents the life support device 100 name, the number “2” represents the life support device 100 version, the portion “L7” represents lot number of the life support device 100 on a panel, the portion “DC” represents bonding operator and the numbers “20140930” represents the starting date of use of the life support device 100.

In order not to cover a big surface area of the life support device 100, the bar code and data matrix may be of small footprint and contain only the necessary data. As described above, the model number and unique serial number of the life support device 100 may be included in the codes. Also, it may be possible to include only data of serial number of the life support device 100, which could be covered with only six characters. With the data included in the codes, other information of the life support device 100 can be obtained from a database. Further, data of multiple experiments, including post analysis, may also be recorded in the database. For example, if it is found that all flies that died or behaved differently are in the same chamber 102a-e, the related results may be disregarded. The serial number can also be used for internal quality control purposes through a database. The database may include information such as date, batch and technician details for every life support device 100 fabricated. The database may be as simple as an excel spreadsheet. It will be appreciated that other types and sizes of bar codes can be used for identifying the life support device 100. Preferably, the code for the life support device 100 should be of smallest possible footprint. A smart phone with bar code scanner function may be used to test whether the size of the code is suitable. In an implementation, 3 codes of smaller sizes may be pasted on the chip.

FIG. 4A shows a perspective view and an exploded view of a life support device 400 for an animal according to a further example embodiment. The life support device 400 comprises a first layer, in the form of top chamber layer 402a and a second layer, in the form of a bottom channel layer 402b. The top chamber layer 402a comprises an aperture 404. The life support device 400 further comprises a groove 406 disposed on the bottom channel layer 402b, forming a fluid channel when the bottom channel layer 402b is bonded to top chamber layer 402a. The life support device 400 further comprises an interconnect 408 and a polydimethylsiloxane (PDMS) valve 410. The interconnect 408 facilitates in easy interfacing of the feed tubes with the life support device 400 and the PDMS valves can be actuated (e.g. pneumatic control) to control the flow of fluid food into the life support device 400.

FIG. 4B shows a perspective view of the life support device 400 and an enlarged view of the fluid channel 412 in FIG. 4A. Here, it is shown that the top chamber layer 402a is covered by a chamber cover 414, forming a chamber 416 with the bottom channel layer 402b for keeping the animal. It is also shown in the enlarged view that the life support device 400 comprises a feeding region 418 for the insertion of a head of the animal at a proximal end of the fluid channel 412. In the embodiment, the fluid channel 412 comprise a paddle shape region 420 adjacent the feeding region 418. It should be noted that the width of the fluid channel 412 at the paddle shape region 420 is the same as the feeding region 418.

FIG. 4C shows a front view and side view of the life support device 400 and the enlarged view of the feeding region in FIG. 4A with example linear dimensions. It is shown that the dimensions of the life support device 400 are 33 mm height and 30 mm width. The dimensions of the chamber 416 are 15 mm height and 20 mm width. The cross-sectional of the fluid channel 412 is rectangular and the dimensions are 0.2 mm width and 0.06 mm-0.1 mm depth. The dimensions of the feeding region are 0.4 mm width and 0.3 mm height. With such configuration, the volume of fluid released from the fluid channel 412 is roughly in the range of 60-100 nl when the animal is fed. However, it will be appreciated that the fluid channel 412 may be of different dimensions. Thus, the volume of fluid food released from the fluid channel 412 would vary according to the dimensions of the fluid channel 412.

FIG. 5 shows images illustrating the steps of fabricating the life support device 400 described in FIGS. 4A-4C. Here, the top and bottom layers are aligned with a L-alignment guide 502 under an inverted microscope. A small amount of acrylic glue is used to bond the two layers at the sides of the layers, to ensure that the layers do not slip. The layers are then sandwiched between a 3 mm thick borosilicate glass pieces and tightened with the help of binder clips. The whole assembly was then placed in a hot air oven for thermal bonding at 125° C. for 45 minutes. The bonded layers are then cooled for another 1 hour. The life support device 400 is then tested with food dyes to check for any leakage. It is noted that the PDMS valve 408 and interconnect 410 in the life support device 400 are recasted from a prefabricated PMMA mould.

FIG. 6A shows images of the feeding region in FIG. 4B when measured with a 3D optical profiler. The fluid channel dimensions and fidelity of the bonding can be tested with a 3D optical profiler, for e.g. Zeta-20, Zeta Instruments and the channel depth of the paddle shape region can be measured with an optical interferometer, for e.g. ContourGT, Bruker. In an embodiment, the 3D optical profiler with objective lens of 40× is used to measure the dimensions of the fluid channel. Table 1 below depicts the averaged actual dimensions of fluid channels for twenty life support devices, before and after bonding.

TABLE 1
Dimensions of fluid channel

| Fluid Channel 1 (Theoretical dimensions, | | | | Fluid Channel 2 (Theoretical dimensions, | | | |
|---|---|---|---|---|---|---|---|
| Depth = 50 μm, Width = 200 nm) | | | | Depth = 100 μm, W = 200 nm) | | | |
| Before Bonding (in μm) | | After bonding (in μm) | | Before bonding (in μm) | | After bonding (in μm) | |
| Channel | Channel | Channel | Channel | Channel | Channel | Channel | Channel |
| width | depth | width | depth | width | depth | width | depth |
| 198.75 +/− 1.51 | 56.7 +/− 3.4 | 181.37 +/− 3.4 | 56.35 +/− 6.27 | 196.28 +/− 1 | 109.7 +/− 4.51 | 173.6 +/− 3.92 | 109.0 +/− 7.86 |

FIG. 6B shows images of the feeding region and paddle shape region in FIG. 4B when measured with ImageJ program. Here, the dimensions and areas of the feeding region and paddle shape region are measured and calculated. The average heights and areas of the feeding region and paddle shape region can be used to calculate the total volume of fluid the regions can hold for feeding. Table 2 below depicts the averaged actual volume of fluid that can be held by the regions of five life support devices. The average volume is found to be 106.11±9 nl, which is very close to the notional volume of 100 nl.

TABLE 2
Volume of fluid held by the feeding region and paddle shape region

| AVERAGE | | | MAX | | | MIN | | | |
|---|---|---|---|---|---|---|---|---|---|
| Channel | Chamber | Total | Channel | Chamber | Total | Channel | Chamber | Total | |
| volume | volume | volume | volume | volume | volume | volume | volume | volume | VARIABILITY |
| 10.33 | 95.781 | 106.111 | 12.213 | 98.406 | 110.619 | 8.447 | 93.156 | 101.603 | 9.016 |

FIGS. 7A and 7B show front view and perspective view of a life support device 700 for an animal respectively according to an example embodiment. Here, the life support device 700 comprises four similar life support devices 702a-d. In the embodiment, the life support device 700 comprises white PMMA piece 704a-b adjacent the fluid channels such that the fluid contained in the fluid channels will have a better contrast with a white background. Each of the fluid channels is in fluid communication with their respective inlet port 706. The top chamber layer 708 and bottom channel layer 710 are fastened together using a magnet 712 and a magnetic housing 714. Apart from that, the structure of the individual life support devices 702a-d has been described in detailed above with respect to FIGS. 4-6. It will appreciated that a life support device may consists a plurality of the individual life support devices 702a-d as described with respect to FIGS. 4-6 and the above configurations in only an example.

FIG. 7C shows a front view of the life support device 700 in FIGS. 7A and 7B with example linear dimensions. As can be seen, the dimensions of the life support device 700 are 72 mm width and 68 mm height. The dimensions of the chambers 716 are 15 mm height and 20 mm width.

FIG. 8A shows an image illustrating examples of various items used to fabricate the life support device in FIG. 7A. A top chamber layer and bottom channel layer are aligned to form a bonded layer 802. One medium glass slide 804 with the dimensions of 50 mm×30 mm, two microscope glass slides 806a, 806b with the dimensions of 25 mm×75 mm, one large glass slide 808 with the dimensions of 50 mm×75 mm are also used for fabricating the life support device. The glass slides above are covered with a yellow Kapton tape to prevent scratching of the bonded layer 802, in the event that the glass slides cracks during the bonding process.

FIG. 8B-8F shows images illustrating the steps of fabricating the life support device 700 in FIG. 7A. As shown in FIG. 8B, it is shown that the bonded layer 802 is placed on the large glass slide 808. As shown in FIG. 8C, the medium glass slide 804 is placed on a recess on the top centre of the bonded layer 802. In FIG. 8D, it is shown that the microscope glass slides 806a, 806b are placed on both sides of the bonded layer 802. The assembly is then tightened by steel clips 810, as shown in FIG. 8E, and placed into a hot air oven at 125° C. for 2 hour. In FIG. 8F, it is shown that UV glue is used to attach needles 812 to the inlets of the bonded layer, completing the process of fabricating the life support device.

FIG. 9 shows a back perspective view of a system 900 for monitoring activity of an animal according to an example embodiment. Here, a breadboard held panel 902 is used to hold a holder panel 904. The holder panel 904 comprises six slots 906 that can fit and secure six life support devices 908a, 908b. The life support devices 908a, 908b used in this embodiment are the same as the life support device as described above with respect to FIG. 1A. The breadboard held panel 902 also hold a camera holder 910 for holding a USB camera (not shown). The breadboard held panel 902 is then stored in an incubator (not shown) at a suitable height to keep the system 900 at constant temperature and humidity. LED lights strip panels (not shown) are located on the top of the incubator. During experiment, the LED lights are set to turn on/off at a designated light/dark cycle.

FIG. 10A shows an image illustrating a back perspective view of a system 1000 for monitoring activity of an animal according to an example embodiment. Here, it is shown that a camera 1002 is held at the camera holder 910. In an embodiment, a camera can video record at least six life support devices 1004, i.e. 30 chambers. The recorded video is then used by a computer program to analyse the height measurements of fluid channels and to determine and record the locations of the animal. In an embodiment, the incubator may store three breadboard held panels 902.

FIG. 10B shows a right side view of the system 1000 of FIG. 10A with example linear dimensions. The length of the breadboard held panel 902 is 450 mm. The distance between a holding member 906 and the camera holder 910 is 392.079 mm. The thickness of the holding member 906 is 32.516 mm. The height of the holder panel 904 is 270 mm. It will be appreciated by a person skilled in the art that the system 1000 may comprise different dimensions and the above-described dimensions are only one of the examples.

FIGS. 11A, 11B and 11C show back view, front view and front perspective view respectively of the system 1000 shown in FIG. 10A. As shown in FIG. 11A, the panel holder 904 secures six life support devices 1102a-e. The panel holder 904 is held on the breadboard held panel 902 with a left side member 1104 and a right side member 1106 secured to the breadboard held panel 902. As shown in FIGS. 11B and 11C, the left side member 1104 comprises a base spring attachment 1108 and a left side front member 1110. The right side member 1106 comprises a spring clamp 1112, a right side front member 1114 and a spring plunger 1116. The left side member 804 and right side member 1106 are used to secure the holder panel 904. Further detail on the base spring attachment 1108, spring clamp 1112 and spring plunger 1116 is described below with respect to FIG. 11D. Also, the camera 1002 is held on a camera holder 910, which is fastened to a camera holder base 1118. In the embodiment, the height of the camera holder 910 can be adjusted along the camera holder base 1118.

FIG. 11D shows an exploded view and configurations of the left side member 1104 and right side member 1106 in FIGS. 11A-110. When placing the holder panel 904 on the breadboard held panel 902, the holder panel 904 is held at an angle and guided into the base of the left side member 1104 and right side member 1106. The base spring attachment 1108 comprises a compression spring 1120 that is disposed within a runner 1122. As the panel is pushed into position, a linear force compresses the spring 1120. Likewise, when the holder panel 904 is taken out, the spring 1120 bounces back to its uncompressed state. The runner 1122 comprises tapered edge for guiding the holder panel 904 in between the left side member 1104 and the right side member 1106. The holder panel 904 is pushed into an upright position and past the spring plunger 1116, which is spring (not shown) loaded. The spring plunger 1116 may reduce vibration and increase the stability of the holder panel 904 when secured in between the left side member 1104 and right side member 1106. The spring clamp 1112 is also configured to secure the holder panel 904 in place. When the holder panel 904 is placed, the spring clamp 1112 is pulled out and twisted from east facing to west facing until it makes contact with the holder panel 904. On the other hand, to get the holder panel 904 out, the spring clamp 1112 is twisted back to east facing and the holder panel 904 is pulled out and past the spring plunger 1116.

FIG. 11E shows an enlarged view of the right hand member 1106 in FIGS. 11A-11D after the holder panel 904 is installed. After placing the holder panel 904, the alignment of the holder panel 904 is checked by making sure that the bottom right hand corner of the holder panel 904 aligns with the side of the right side member 1106.

FIGS. 12A, 12B and 12C show back view, front view and exploded view respectively of a slot 1200. As shown in FIG. 12A, the slot 1200 comprises a spring ultra low screw disposed within a spring holder 1204. The slot 1200 further comprises a side spring holder 1206, abutting the spring holder 1204. A first flat spring (not shown) is attached to the side spring holder 1206. In FIG. 12C, it is illustrated that the slot further comprises the first flat spring 1208 and a second flat spring 1210. When placing the life support devices 1212 into the slot 1200, the life support device 1212 is placed on the holder panel 1214 such that the back surface of the life support device 1212 flush with the holder panel 1214. The life support device 1212 is then pushed vertically into the slot 1200, by applying a force to the front surface of the life support device 1212. As the life support device 1212 is pushed into the slot 1200, the first flat spring 1208 and second flat spring 1210 apply force to the life support device 1212 in both the x and z axes. When taking the life support device 1212 out from the slot 1200, the life support device 1212 is pulled vertically upward until it is released from the slot 1200.

FIG. 12D shows a back view of the slot 1200 in FIG. 12A-12C after the life support device 1212 is installed. When placing the life support device 1212 into the slot 1200, the alignment of the life support device 1212 is checked by making sure that the bottom left hand corner of the life support device 1212 aligns with the side of the spring holder 1204 and the top right hand edge of the life support device 1212 aligns with a reference line 1216.

FIG. 13A shows an image illustrating a holder panel 1300 and six life support devices 1302a-f during an experiment according to an example embodiment. Here, all the life support devices 1302a-f are held by slots 1304. Each chamber 1306 of the life support devices 1302a-f contains a fly and the fluid channels 1308 are filled with fly media and/or water.

FIG. 13B shows an image illustrating an interface of an example computer program suitable for use in an example embodiment. In this example, a computer program, GUI MATLAB is used to analyse a video file recorded by a camera in an experiment. The program is configured to calculate liquid levels of each fluid channels 1308 and the locations of the flies in each chamber 1306. The computer program is also configured to select a region of interest (ROI) 1310. The data of the experiment can be illustrated in various forms, for e.g. graphs and tables.

FIG. 14 shows an image illustrating a system 1400 for monitoring activity of an animal and an enlarged view of the life support device according to an example embodiment. The system is used to conduct eight separate behaviour experiments, one on each individual life support device 1402. The life support devices 1402 used in this embodiment is the life support device 1402 as described above with respect to FIG. 4A. The experiments are conducted inside the incubators, for e.g. MIR-154, Sanyo, where the temperature in the incubator can be controlled manually. Four life support devices 1402 are placed on top of the LCD screen 1404, for e.g. uLCD-43, 4D systems, where light stimuli may be provided. Two LCD screens 1404 are fixed on an aluminium panel below the life support devices 1402. A speaker 1406 is placed next to the LCD screens 1404 to provide sound stimuli. Eight pumps 1408 are attached to the incubator wall to supply fluid to the inlets of the life support devices 1402. The system 1400 further comprises white LED strips, for e.g. ST-6500-CT, Inspired LED, which illuminates the incubator chamber at about 600 lux, measured on the surface of the life support devices 1402. In addition, two FireWire colour cameras 1410, e.g. A601fc Basler, are held in the incubator to monitor the activity of the animal and fluid location in the life support devices 1402.

FIG. 15 shows an image illustrating an interface of an example computer program used to control the system 1400 in FIG. 14. In this example, the computer program used is LabVIEW program. The program is configured to control various activities in the incubator. For example, the program is configured to time the feeding of the animal in the chambers and changing the light conditions in the incubator. The program is also configured to display and analyse the video recorded by the cameras.

FIGS. 16A and 16B show a perspective view of a refilling device 1602 and an exploded view of the refilling device 1602 respectively according to an example embodiment. The refilling device 1602 comprises a housing 1604 which is adapted to receive the life support device 1606. The refilling device 1602 further comprises a clamp 1608 that is connected to the housing 1604. The clamp 1608 is operable to secure the life support device 1606 in the housing 1604. The refilling device 1602 also comprises at least one fluid conduit, represented as tube 1610, attachable to the housing 1604 for discharging fluid into the fluid channels of the life support device 1606. As shown in FIG. 16A, the housing 1604 comprises taper edged member 1612 on both sides of the housing 1604 for guiding the life support device 1606 into the housing 1604. It should also be noted that the housing 1604 comprises transparent covers 1614a, 1614b separated by a transparent silicone mat 1614c. In an embodiment, the clamp 1608 is connected to the housing 1604 using a biasing means, represented as spring 1616. When placing the life support device 1606 into the housing 1604, the clamp 1608 may be pulled and the life support device 1606 is inserted into the housing 1604. The clamp 1608 is then released, pushing the bottom part 1618 of the life support device 1606 upward to secure the life support device 1606 in place.

FIGS. 16C and 16D show images illustrating the arrangement of various items used to refill the life support device 1606 in FIG. 16A and the refilling device 1602 in FIG. 16A when used to refill the life support device 1606 respectively. When refilling the life support device 1606, needles 1620 (FIG. 16B) attached to the tubes 1610 are aligned to prepare for receiving the fluid channels of the life support device 1606. The tubes 1610 are placed into a media bottle 1622. The tubes 1610 are then pushed on a silicone mat 1614c of the housing 1604. The tubes 1610 are placed in the housing 1604 and clamped using a bar 1624 using screws 1626 attached to the housing 1604. The tubes 1610 are locked to one or more pump 1628. A dummy device 1606 is inserted into the housing 1604 and the clamp 1608 is pulled, such that all the needles 1620 of the tubes 1610 are aligned. The pump 1628 is turned on to flush some liquid through the tubes 1610 to make sure that all the tubes 1610 are filled with the liquid. The channels of the dummy device are checked to make sure that each channel is filled simultaneously. The dummy device 1606 is then replaced with a life support device 1606 and the pump 1628 is actuated. The flowing rate of the liquid can be controlled by adjusted the bar by turning the screws 1626

### Experimental Data 1

Using the device and systems illustrated in the example embodiments, experiments have been carried out using the life support device described in FIG. 1A. The subjects used in the experiment were out-crossed with CS strain for five generations. Four Gal4 drivers: TH-Gal4, Tdc2-Gal4, Trh-Gal4 and CS-Gal4 flies-were crossed with UAS-Kir2.1; tub-Gal80ts to produce F1 progenies used for the behavioural experiments. F1 progenies were maintained at a temperature of 22° C., 60-70% relative humidity, 12-hour light-dark cycles for 4 to 7 days before the experiment day. Kir2.1 was expressed by incubating the flies at 31° C. for 24 hours. Flies were allowed to readjust at 22° C. for 24 hours before the experiments. Fly groups were starved for 24 hours with a strip of filter paper soaked in deionized water before the experiments are carried out, unless they are fed. Red fluid with 5% sucrose and liquid food consisted of ingredients listed on Table 3 below were loaded into the fluid channels before loading the flies. The experiment was recorded and analysed off-line.

TABLE 3
List of ingredient used for liquid food

| Ingredients | Mass to Add (per 10 ml DI H₂O) | |
|---|---|---|
| Yeast Extract | 0.4 | g |
| Peptone | 0.2 | g |
| Sucrose | 0.3 | g |
| Glucose | 0.6 | g |
| MgSO₄ × 6 H₂O | 0.005 | g |
| CaCl₂ × 2 H₂O | 0.005 | g |

| | |
|---|---|
| Distilled Water | Add to above to make 10 ml. |

The life support devices were loaded with flies and were mounted on the holder panel inside an incubator. The camera video record recorded the activities of the flies after the holder panels were secured. The recording was carried out for 24 hours. The recorded video file was then saved in windows media video format at 512 kbps and video size of 720p HD standard resolution. Flies were discarded after the experiment.

Drosophila behaviour was analysed through the recorded video file. An MATLAB program as described above with respect to FIG. 13B was used to analyse the height of fluid in the channels and locations of Drosophila. The fluid level was measured using colour as a marker. The program observed the point where colour changed vertically along the selected region. Location of the fly was a centroid of the 2-Dimensional image of the behaviour chambers. The background was white, while a fly body colour is black. An eight-connected image segmentation method was used to classify the object. With this method, more than one fly can be tracked at the same time in the chambers.

The feeding time and volume were computed from the arrays of fluid positions. After the positions were tracked, the shifts in the fluid position were observed using an algorithm. The algorithm sums the amount of fluid when the positions decrease for every 5 second. If the fluid reduces more than a threshold level, the shift is considered as feeding. The threshold was set at 0.5 mm in the setting. Total volume is equal to sum of all portions volume.

FIG. 17 shows graphs illustrating survival rate and climbing assay results of the experiment according to an example embodiment. It is shown that in the experiment that flies could survive on liquid food for at least 5 days with only one fly deceased. The food formula as listed in Table 3 showed higher survival rate than 5% sucrose solution. The climbing assay did not show any deficit in climbing ability of Drosophila. Therefore, the liquid food can be used as a food source for long-term experiments or at least for a 5-day period.

FIGS. 18A and 18B show graphs illustrating the results of the experiment including the fluid measurements of channels and locations of flies over time according to an example embodiment. Two examples are shown in FIGS. 18A and 18B respectively. The computer program provides data related to fluid measurements of the channels and locations of the flies. In FIGS. 18A and 18B, the locations of the fly were given in two graphs, i.e. vertical position graph 1802a and horizontal position graph 1802b. It can be seen from these graphs that in 5 hours, there were periods in which flies continuously moved up and down along the chambers. Inactivity period as long as 10 to 30 minutes can also be observed. The shaded part of the vertical position graph 1802a indicates that the fly was at the top third part of the chambers. In the experiment, an event may only be considered as feeding period when the vertical position graph 1802a is indicated as shaded. Otherwise, the event will be ignored.

FIG. 18C shows an enlarged view of a graph illustrating the fluid measurements of a channel according to an example embodiment. The height of the fluid was measured and plotted against time. The graph shows two feedings observed within a 5 hour experiment as there were two sudden reductions 1804a, 1804b in the level of fluid within a short period of time. Both feedings have approximately the same volume. The jitter noise level is well below the feeding level and can be removed by setting an appropriate feeding threshold value. The linear slope along the shift in location of fluid over time indicates low evaporation rate. As described above, the amount of fluid taken by a single Drosophila can be accurately timed and quantified.

FIG. 18D shows graphs illustrating results of the experiment comparing feeding behaviour of starved and non-starved subjects according to an example embodiment. The experiment was carried out to study feeding behaviour. CS flies which were starved for 24 hours prior the experiment was compared with non-starved CS flies during a 5 hour experiment. In FIG. 18D, Graph A illustrates the total amount of feeding of each fly, Graph B illustrates the number of portions each fly consumed during experiment; Graph C illustrates the time points the fly fed, Graph D illustrates the first feeding time, Graph E illustrates the time spend feeding from the channel, Graph F illustrates the volumes of the single feeding portions. It is observed that starved flies fed on average 0.09 μl more than non-starved flies (0.16 μl±0.05 vs 0.07 μl±0.03; Graph A), spending less time at the feeding ports (83 s±6 vs 363 s±73; Graph E) and consuming more and bigger portions (portion number: 2±0.05 vs 1.13±0.5; Graph B, feeding portion volume: 0.08 μl±0.008 vs 0.06 μl±0.008; Graph F). Feeding time and first feeding time were similar in both groups with the mean feeding time 1.88 hr±0.34 vs 1.6 hr±0.5 (Graph C) and 1.27 hr±0.46 vs 1.22 hr±0.6 (Graph D) subsequently.

FIG. 18E shows a graph illustrating the results of the experiment comparing influence of candidate neurotransmitters on feeding behaviour of flies according to an example embodiment. Candidate neurotransmitters (octopamine —tdc2, dopamine —TH, and serotonin—Trh) known to influence feeding were tested for their role in Drosophila feeding behaviour. Conditional expression of Kir2.1 was used to silent the specific set of neurons. When flies were raised at permissive temperature (no incubation is indicated as “no inc” in FIG. 18E), Gal80ts was expressed, inhibiting expression of Kir2.1. When flies were shifted to restrictive temperature for 24 hour (incubation is indicated as “inc” in FIG. 18E), Gal80ts was inactive, allowing expression of Kir2.1, resulting in the neurotransmitter deficiency. It is found that dopamine-deficient flies showed similar feeding to control fluid consumption upon 24 hour starvation under both conditions (0.11 μl±0.4). However, octopamine deficient flies (Tdc2 inc) showed reduced feeding (0.33 μl±0.08 vs 0.14 μl±0.04) and flies that lack serotonin (Trh inc) showed increased feeding (0.11 μl±0.04 vs 0.23 μl±0.08). This result allows the scientists to pursue the studies focusing on these two neuromodulators and continue the screening with other neurotransmitters that could affect feeding.

### Experimental Data 2

Drosophila melanogaster, four to seven day-old males and females were separated and transferred to the empty vials for starvation (8-10 flies per vial) 24 hours prior the experiments to increase their motivation to feed. All the flies were maintained at 12:12 hour light and dark cycle at 22° C. during starvation period. Flies were anesthetized on ice for less than one minute and transferred individually to separate chips. Upon loading, flies were allowed to habituate for 5 minutes before the experiments were started.

FIG. 19 shows a chart illustrating the experiment trials that are carried out according to an example embodiment. Each group of flies was subjected to six training trials (Pavlovian conditioning) with two minutes inter-trial interval. In the Sham trial, a 300 Hz sound stimulus overlapped with a blue-light screen change. Food was visible but not reachable for the fly. In the Stimuli experiments, flies were given either sound, light stimuli or none, but each trial contains the food reward. In the control experiments light, sound and food were given.

Blue light was kept until flie's head was detected at the feeding chamber. The screens were switched to white colour. Food was instantly retracted upon completion of the trial or detection of a fly head in the food port or 100 second. Each trial was separated by the 120 seconds interval. To test the importance of the each stimuli, separate and together, experiments were performed in which only one of the cues, both or none were given. In Sham trials, animals were given both stimuli, with the food presented/visible but not reachable for the fly. All experiments were conducted in 25° C. Training and test sessions were recorded on video for later analysis. The number of successful trials, time period and travelling path were used to measure learning/feeding performance.

Animal location and orientation were tracked in real time. The region of each behaviour chamber was extracted and analysed individually. Either red or blue colour plane was extracted from recording images when the LCD screen was white or blue, consecutively. Then an appropriate threshold value was set to convert the extracted colour plane into the binary image separating the animal body and background. The centre of gravity was computed as the location of the animal. The pixel scale was converted into millimetre scale before further analysis. The animal orientation was computed by forming the line between the farthest two pixels representing the animal body.

Pre-trial activity was monitored during the first ten minutes period of the experiment. The location and speed were recorded within selected region. Time spent on a location within a behaviour chamber was computed by counting the number of frames an animal located within each 1 mm×1 mm area. The heat map showed the normalized scale of time spent at each position.

Task performance is the number of trials the animal fed on the fluid inside the feeding region divided by the total number trial. Time to feed is the time that the animal spent between signal given and head detected inside the chamber. Path performance is the ratio between the distance used and the shortest possible distance. Path performance data is transformed with Box-Cox method before statistical tests. Time to feed and path performance were only computed on performed trials. Correlation between path performance and time to feed was measured as the slope of linear regression. Time spent at the feeding region is the time the animal spent within 3 mm around the feeding region 3 minutes after the animal is fed (30 seconds after head detect within feeding region).

All data were tested for normality by Shapiro-Wilk test. All the data did not pass the normality test. The behaviour data were plotted in bar graph with confident interval (CI) as error bar and reporting value, except that effective path ratio was shown in scatter plot. Mann-Whitney U-tests and Kruskal-Wallis tests were used for pair-wise and between-group comparisons. Hedge-g (g) and area under the curve (AUC) were reported as the effect size for pair-wise comparisons. Eta square (η²) was reported as the effect size for between-group comparison. A significant level (α) was set at 0.05. All sample sizes were reported as subscript.

FIG. 20 shows graphs illustrating experimental results conducted on four wild types flies according to an example embodiment. In the experiment, four widely used wild-type drosophila, namely, CS, OR, w1118, and yw were tested in our stimuli and feeding association assay. These flies, in different genetic background, were kept at the laboratory and starved for 24 hours before the experiments. All the wild types flies performed the feeding task by moving toward the feeding region to obtain fluid reward and performed the task at the same performance level, i.e. near 60 percent (p=0.094, χ²_{3,481}=6.38, η²=0.013; 62.15±4.67% CS, 54.69±5.48% OR, 64.4±3.55% w1118, and 62.43±5.53% yw; FIG. 20A). On average, All the wild types flies first went to feed on reward within the first two trials (p=0.002, χ²_{3,481}=14.23, 6²=0.021; 1.80±0.71 trials CS, 2.21±0.3 trials OR, 1.58±0.22 trials w1118, and 1.91±0.26 trials yw; FIG. 20B).

The time the flies required to forage to the food after stimuli given were different across all wild-types (p=1.33E-5, χ²_{3,1752}=25.3, p²=0.0144; FIG. 20E). OR-type flies spent more time (34.01±2.56 s) to find reward than the others on average from all trials (p=0.009, U_{420,496}=113E3, AUC=0.55, g=0.173 vs CS; p=3.6E-4, U_{420,398}=72E3, AUC=0.43, g=0.23 vs w1118; p=1.86E-6, U_{420,442}=109E3, AUC=0.59, g=0.29 vs yw). The change in time to feed after performing each success trail was also recorded and analysed. Time to feed decreased after each success as observed in the first three success trials (33.21±4.43 s in the 1^{st} trial, 29.19±4.81 s in the 2^{nd} trial, and 28.36±4.73 s in the 3^{rd} trial in CS; FIG. 20F). However, no large change occurred across all success trials (p=0.31, χ²_{3,496}=5.93, r²=0.012 CS; p=0.55, χ²_{3,420}=4.0, η²=0.01 OR, p=0.09, χ²_{3,398}=9.66, R²=0.024 w1118, p=0.70, χ²_{23,442}=3.01, η²=0.007 yw).

The paths from the start location after the stimuli are given to the feeding location were analysed to compare the differences in path choices among the wild types. The total distance travel per trial varied among all wild-types (p=1.42E-63, χ²_{3,1752}=294.66, η²=0.168; 52.06±5.51 mm CS, 239.9±39.07 mm OR, 683.89±39.07 mm w1118, and 235.77±40.4 mm yw; FIG. 20D). CS-type flies travelled with the shortest distance per trial (p<1E-80, U_{496,419}=145E3, AUC=0.7, g=0.67 vs OR; p<1E-80, U_{496,95}=160E3, AUC=0.82, g=0.86 vs w1118; p<1E-80, U_{496,442}=149E3, AUC=0.6, g=0.54 vs yw). OR-type flies and yw-type flies travelled with similar distance per trial (p=0.27, U_{419,442}=94E3, AUC=0.51, g=0.01). The effective path ratio measures how well flies made their path. In other words, the higher the effective path ratio, the better the path. The effective path ratio also varied among all wild types (p=1.52E-52, χ²_{3,1752}=293.68, η²=0.168; 0.306±0.02 CS, 0.187±0.02 OR, 0.12±0.016 w1118, and 0.193±0.021 yw; FIG. 20C). CS-type flies had the highest effective path ratio (p<1E-80, U_{496,419}=65E3, AUC=0.31, g=0.55 vs OR; p<1E-80, U_{496,395}=41E3, AUC=0.21, g=0.92 vs w1118; p<1E-80, U_{496,442}=684E3, AUC=0.31, g=0.5 vs yw). Other flies chose the less effective path as can be seen from the high number of samples gathering at the lower effective path ratio value (FIG. 20C). It appears that all tested wild-type drosophila performed the feeding task at the same level of performance, but behaved differently in path choice and time spent.

FIG. 21 shows graphs illustrating analysis results for four wild types flies in a ten minutes pre-trial period according to an example embodiment. Before the experiment, the flies were given 10 minutes to habituate to a new environment inside the chip. Fly behaviour was observed, including location and speed. Activity index shows that the flies were active on average 35.9 percent during the pre-trial period (35.9±3.58% CS, 27.22±3.21% OR, 48.93±4.97% w1118, and 34.12±3.79% yw; FIG. 21B). The activity are different among wild types (p=7.88E-10, χ²_{3,476}=45.33, η²=0.095). The average speed were 1.53 mm/s (1.45±0.17 mm/s CS; 0.93±0.12 mm/s OR; 2.91±0.29 mm/s w1118; 1.1±0.14 mm/s yw; FIG. 21A). w1118-type flies were significantly more active and moved faster than other wild types (activity index: p=4.71E-5, U_{100,133}=8639, AUC=0.56, g=0.56 vs CS; p=9.52E-11, U_{100,127}=9479, AUC=0.75, g=0.99 OR; p=9.09E-6, U_{100,117}=7826, AUC=0.67, g=0.63 yw; speed: p=9.99E-16, U_{100,123}=10E3, AUC=0.80, g=1.17 vs CS; p<1E-80, U_{100,127}=11E3, AUC=0.90, g=1.79 vs OR; p<1E-80, U_{100,117}=10E3, AUC=0.88, g=1.57 vs yw). This demonstrates that flies behaved differently, depending on their genetic background. All flies moved slower as they started to habituate to the new environment. The mean speeds during the last half-minute epoch were lower than the last epoch (2.07±0.22 to 1.64±0.45 mm/s CS, 1.52±0.23 to 1.13±0.16 mm/s OR, 3.8±0.46 to 2.98±0.42 mm/s w1118, 2.15±0.3 to 1.1±0.15 mm/s yw).

The time spent on each location (1×1 mm) was calculated during the 10-minute pre-trial period. Selectivity and entropy of time spending matrices were compared to measure the different in behaviour between wild types. The difference in selectivity and entropy indicate the different in new environment exploration pattern among wild types (selectivity p=6.17E-5, χ²_{3,476}=22.11, η²=0.046; entropy p=1.3E-3, χ²_{3,476}=15.68, η²=0.032; FIGS. 21C, 21D). CS-type flies showed similar value of selectivity (p=0.207, U_{113,117}=7E3, AUC=0.47, g=0.13) and entropy (p=0.49, U_{113,117}=7E3, AUC=0.5, g=0.08) with yw, but not with OR (selectivity: p=3.1E-3, U_{113,127}=10E3, AUC=0.6, g=0.09; entropy: p=0.02, U_{113,127}=7E3, AUC=0.43, g=0.18) and w1118 (selectivity: p=0.04, U_{113,100}=5E3, AUC=0.43, g=0.27; entropy: p=0.02, U_{113,100}=7E3, AUC=0.58, g=0.08). From the experiment, it is inferred that pre-trial behaviours were different across wild-type strains.

FIG. 22 shows graphs comparing experimental results for a default experiment and a sham experiment according to an example embodiment. To demonstrate whether reward presence, stimuli, and other cues affect the performance of the task, the sham experiment was designed. In the sham experiment, the fluid reward was driven along the channel after stimuli were given, but the fluid was stopped at the outlet before the feeding region where flies could not access with proboscis. The results showed that without reward, the performance is relatively lower (p<1E-80, U_{113,49}=5.5E2, AUC=0.0858, g=1.91; 13.61±4.92% no reward and 62.16±4.67% with reward; FIG. 22A) and the first success trial is significantly higher for experiments with no reward than experiments with reward presented (p<7.75E-10, U_{113,49}=5E3, AUC=0.79, g=1.39; 4.04±0.57 trials no reward and 1.8±0.24 trials with reward; FIG. 22B). If the flies performed because of reward, the performance should be higher in sham experiment. Thus, it appears that the presence of reward was not sufficient for the flies to perform the task.

FIG. 23 shows graphs comparing experimental results conducted for three different temperatures settings according to an example embodiment. As shown in FIG. 23, The temperature affects the path the flies approached the reward (effective pat, and ratio: p=2.11E-63, χ²_{3,1109}=288.62, η²=0.26; 0.15±0.011 18C, 0.306±0.01 25C, and 0.101±0.009 31C; total path distance: p=3.31E-74, χ²3,1109=338.39, η²=0.31; 295.8±27.28 mm 18C, 52.06±5.51 mm 25C, and 719.16±110.64 mm 31C; FIGS. 23C-D). Both low and high temperature environments significantly decrease the effective path ratio (p<1E-80, U_{496,298}=38E3, AUC=0.26, g=−0.72 18C vs 25C; p<1E-80, U_{496,316}=27E3, AUC=0.17, g=1.01 25C; 25 vs 31C) and total path used per trial (p<1E-80, U₇₉₄=112E2, AUC=0.76, g=0.83 18C vs 25C; p<1E-80, U₈₁₂=133E3, AUC=0.85, g=−1.06 25C; 25 vs 31C).

However, it was noted that time to feed did not change over change in temperature (p=0.18, χ²_{3,1115}=3.47, η²=0.003; 31.4±2.74 s 18C, 29.51±2.2 s 25C, and 31.6±2.72 s 31C; FIG. 23E). Correlation between the effective path ratio and time to feed decreased as temperature shifted from 25C (r=0.245 18C, r=0.59 25C, and r=0.321 31C). The flies could perform the task at the same level (p=0.18, χ²_{3,306}=3.43, η²=0.011; 59.83±6.56% 18C, 62.16±4.67% 25C, and 58.97±5.81% 31C; FIG. 23A) and the same number of trials to perform the first success feeding (p=0.21, χ²_{3,306}=3.12, η²=0.01; 2.06±0.33 trials 18C; 1.8±0.24 trials 25C; 2.04±0.32 trials 31C; FIG. 23B) over three different temperature settings. This indicates that this task can be used at different temperature settings. However, the behaviour can only be compared within the same temperature settings. It was thus found that the foraging paths that were used by the flies were affected by the temperature settings, but the performance was not affected by the temperature.

FIG. 24 shows graphs comparing experimental results conducted for four different stimuli conditions according to an example embodiment. In this experiment, it is demonstrated that flies uses the stimuli to search for the rewards. Four experiments with different stimuli protocols were conducted-both blue light and 300 Hz tone sound, sound only, light only, and none. With one or both stimuli absent, the performance was lower (p=1.33E-5, χ²_{3,441}=25.32, η²=0.058; 62.16±4.67% both light and sound, 46.73±5.16% sound only, 51.43±6.79% light only, and 44.5±5.41% none; FIG. 24A), and a number of trials before the first success feeding was higher (p=4.38E-3, χ²_{3,441}=13.12, η²=0.03; 1.8±0.24 trials both light and sound, 2.25±0.33 trials sound only, 2.58±0.36 trials light only, and 2.13±0.33 trials none; FIG. 24B). No difference in performance was observed between scenarios when either sound or both stimuli were absent (p=0.076, U_{105,97}=4.4E3, AUC=0.44, g=−0.22). The results showed that flies could use light as a cue better than sound (p=1.64E-5, U_{107,105}=6E3, AUC=0.54, g=0.148).

Other than performance, the total path (p=2.84E-35, χ²_{3,1378}=163, η²=0.119; 52.06±5.51 mm both light and sound, 76.73±10.42 mm sound only, 47.52±5.86 mm light only, and 391.52±75.7 mm none; FIG. 24D) and effective path ratio (p=1.84E-39, χ²_{3,1378}=183.15, η²=0.13; 0.31±0.02 both light and sound, 0.28±0.03 sound only, 0.29±0.03 light only, and 0.14±0.02 none; FIG. 24C) were also worsened. Without any stimulus, flies could not time the reward, as a result, total path was significantly more (p<1E-80, U_{496,259}=99E3 AUC=0.77, g=0.93 vs both sound and light; p<1E-80, U_{324,259}=60E3, AUC=0.71, g=0.75 vs sound only; p<1E-80, U_{300,259}=60E3, AUC=0.78, g=0.81 vs light only). Without any stimulus, time to feed slightly increased (p=2.7E-3, χ²_{3,1378}=14.15, η²=0.01; 29.51±2.2 s both light and sound, 32.14±2.84 s sound only, 32±2.95 s light only, and 26.42±3.22 s none; FIG. 24E).

FIG. 25 depicts an exemplary computing device 2500, hereinafter interchangeably referred to as a computer system 2500, suitable for implementing at least some steps of the method described in the example embodiments. The following description of the computing device 2500 is provided by way of example only and is not intended to be limiting.

As shown in FIG. 25, the example computing device 2500 includes a processor 2504 for executing software routines. Although a single processor is shown for the sake of clarity, the computing device 2500 may also include a multi-processor system. The processor 2504 is connected to a communication infrastructure 2506 for communication with other components of the computing device 2500. The communication infrastructure 2506 may include, for example, a communications bus, cross-bar, or network.

The computing device 2500 further includes a main memory 2508, such as a random access memory (RAM), and a secondary memory 2510. The secondary memory 2510 may include, for example, a hard disk drive 2512 and/or a removable storage drive 2514, which may include a floppy disk drive, a magnetic tape drive, an optical disk drive, or the like. The removable storage drive 2514 reads from and/or writes to a removable storage unit 2518 in a well-known manner. The removable storage unit 2518 may include a floppy disk, magnetic tape, optical disk, or the like, which is read by and written to by removable storage drive 2514. As will be appreciated by persons skilled in the relevant art(s), the removable storage unit 2518 includes a computer readable storage medium having stored therein computer executable program code instructions and/or data.

In an alternative implementation, the secondary memory 2510 may additionally or alternatively include other similar means for allowing computer programs or other instructions to be loaded into the computing device 2500. Such means can include, for example, a removable storage unit 2522 and an interface 2520. Examples of a removable storage unit 2522 and interface 2520 include a program cartridge and cartridge interface (such as that found in video game console devices), a removable memory chip (such as an EPROM or PROM) and associated socket, and other removable storage units 2522 and interfaces 2520 which allow software and data to be transferred from the removable storage unit 2522 to the computer system 2500.

The computing device 2500 also includes at least one communication interface 2524. The communication interface 2524 allows software and data to be transferred between computing device 2500 and external devices via a communication path 2526. In various embodiments of the inventions, the communication interface 2524 permits data to be transferred between the computing device 2500 and a data communication network, such as a public data or private data communication network. The communication interface 2524 may be used to exchange data between different computing devices 2500 which such computing devices 2500 form part an interconnected computer network. Examples of a communication interface 2524 can include a modem, a network interface (such as an Ethernet card), a communication port, an antenna with associated circuitry and the like. The communication interface 2524 may be wired or may be wireless. Software and data transferred via the communication interface 2524 are in the form of signals which can be electronic, electromagnetic, optical or other signals capable of being received by communication interface 2524. These signals are provided to the communication interface via the communication path 2526.

As shown in FIG. 25, the computing device 2500 further includes a display interface 2502 which performs operations for rendering images to an associated display 2530 and an audio interface 2532 for performing operations for playing audio content via associated speaker(s) 2534.

As used herein, the term “computer program product” may refer, in part, to removable storage unit 2518, removable storage unit 2522, a hard disk installed in hard disk drive 2512, or a carrier wave carrying software over communication path 2526 (wireless link or cable) to communication interface 2524. Computer readable storage media refers to any non-transitory tangible storage medium that provides recorded instructions and/or data to the computing device 2500 for execution and/or processing. Examples of such storage media include floppy disks, magnetic tape, CD-ROM, DVD, Blu-ray™ Disc, a hard disk drive, a ROM or integrated circuit, USB memory, a magneto-optical disk, or a computer readable card such as a PCMCIA card and the like, whether or not such devices are internal or external of the computing device 2500. Examples of transitory or non-tangible computer readable transmission media that may also participate in the provision of software, application programs, instructions and/or data to the computing device 2500 include radio or infra-red transmission channels as well as a network connection to another computer or networked device, and the Internet or Intranets including e-mail transmissions and information recorded on Websites and the like.

The computer programs (also called computer program code) are stored in main memory 2508 and/or secondary memory 2510. Computer programs can also be received via the communication interface 2524. Such computer programs, when executed, enable the computing device 2500 to perform one or more features of embodiments discussed herein. In various embodiments, the computer programs, when executed, enable the processor 2504 to perform features of the above-described embodiments. Accordingly, such computer programs represent controllers of the computer system 2500.

Software may be stored in a computer program product and loaded into the computing device 2500 using the removable storage drive 2514, the hard disk drive 2512, or the interface 2520. Alternatively, the computer program product may be downloaded to the computer system 2500 over the communications path 2526. The software, when executed by the processor 2504, causes the computing device 2500 to perform functions of embodiments described herein.

It is to be understood that the embodiment of FIG. 25 is presented merely by way of example. Therefore, in some embodiments one or more features of the computing device 2500 may be omitted. Also, in some embodiments, one or more features of the computing device 2500 may be combined together. Additionally, in some embodiments, one or more features of the computing device 2500 may be split into one or more component parts.

It will be appreciated that the elements illustrated in FIG. 25 function to provide means for performing the various functions and operations of the servers as described in the above embodiments.

In an implementation, a server may be generally described as a physical device comprising at least one processor and at least one memory including computer program code. The at least one memory and the computer program code are configured to, with the at least one processor, cause the physical device to perform the requisite operations.

The device methods and systems described above can be used for various other applications. For example, the design of two feeding channels for a single chamber allows more experiments options according to a researcher's choice of feeding preference studies. The life support device as disclosed is easy to refill or replace, allowing it to be used in feeding-sleeping studies, where flies circadian rhythm is monitored for a long period of time. Further, since the assay can provide many behavioural metrics within one experiment, it may also be used for drug screening, where the researcher is able to control the drug dose, concentration, and time of delivery to the animal. In other words, this device can be used in many areas, including drug screening, feeding disorders and sleep studies.

Further, the life support device as disclosed in the example embodiments, integrated with microfluidic technology can dispense 60-100 nm of fluid into the chamber at multiple (e.g. 6-12) times, with the fluid actively controlled throughout an experiment. This allows for long-term fly feeding for at least 1-2 days. This also allows various experiments to be conducted to study the reaction of an animal with respect to reward. The life support device can be simple in handling, reliable and information-rich. It can be also be used to study biological hypothesis, as well as a behavioural drug-screening platform. For example, a new feeding assay using Drosophila would allow research to be carried out on memory, attention and feeding deficiency of the Drosophila. The results can be used as a guideline on the effect of test drug on a disease. Behaviour results can be used as a guideline to show the effect of test drug on certain disease.

The life support device as disclosed in the example embodiments allows the levels of fluid in the channels to be measured through the video data, as well as animal activity to be tracked simultaneously. The platform allows probing into several basic behaviours of the animals such as walking, sleeping, mating, foraging and feeding. Knowing the fluid level during the period of experiment also allows determining the precise feeding time, feeding bout and feeding amount for an animal. The microfluidic channel is also able to accurately monitor a miniscule meal size of about 0.08 ul per meal on average.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

What is claimed is:

1. A method of monitoring activity of an animal, the method comprising the steps of:
retaining the animal in an enclosed chamber;
controlling a food supply to the animal in the enclosed chamber as a first stimulus; and
monitoring the activity of the animal in response to the first stimulus,
wherein monitoring the activity of the animal comprises analysing change in a volume of food in the food supply and location of the animal in the enclosed chamber over a predetermined period of time.

2. The method as claimed in claim 1, wherein controlling the food supply to the animal comprises dispensing the food via a microfluidic channel, the microfluidic channel having predetermined dimensions.

3. The method as claimed in claim 2, wherein controlling the food supply further comprises operating a pump attached to the microfluidic channel.

4. The method as claimed in claim 2, wherein analysing the change in the volume of food comprises measuring height of a fluid containing the food in the microfluidic channel over the predetermined period of time.

5. The method as claimed in claim 1, wherein analysing the location of the animal over the predetermined period of time comprises obtaining video data of the animal using an imaging device and extracting positional information of the animal from the video data.

6. The method as claimed in claim 5, wherein extracting positional information comprises determining a position of centroid of the animal body.

7. The method as claimed in claim 5, wherein extracting positional information comprises determining an orientation of the animal.

8. The method as claimed in claim 5, further comprising controlling the food supply to the animal based on the positional information of the animal.

9. The method as claimed in claim 1, further comprising:
providing a second stimulus different from the first stimulus to the animal; and
monitoring the activity of the animal in response to a combination of the first and second stimulus.

10. The method as claimed in claim 9, wherein the second stimulus comprises one selected from a group consisting of a light, a sound, an electric current, an odour and a temperature change.

11. A system for monitoring activity of an animal, the system comprising:
an enclosed chamber for retaining the animal;
a food supply for feeding the animal to provide a first stimulus; and
monitoring means configured to monitor the activity of the animal in response to the first stimulus;
wherein the monitoring means is configured to analyse change in a volume of food in the food supply and location of the animal in the enclosed chamber over a predetermined period of time for monitoring the activity of the animal.

12. The system as claimed in claim 11, wherein the food supply is configured to dispense the food via a microfluidic channel in fluid communication with the enclosed chamber, the microfluidic channel having predetermined dimensions.

13. The system as claimed in claim 12, further comprising a pump attached to the microfluidic channel and operable to supply food to the microfluidic channel.

14. The system as claimed in claim 12, wherein an end of the microfluidic channel adjacent to the enclosed chamber comprises a feeding region, the feeding region configured for access by the animal.

15. The system as claimed in claim 12, wherein the monitoring means is configured to measure a height of a fluid containing the food in the microfluidic channel over the predetermined period of time for analysing the change in the volume of the food.

16. The system as claimed in claim 11, wherein the monitoring means is configured to obtain video data of the animal using an imaging device and extract positional information of the animal from the video data for analysing the location of the animal over the predetermined period of time.

17. The system as claimed in claim 16, wherein the positional information comprises a position of a centroid of the animal body.

18. The system as claimed in claim 16, wherein the positional information comprises an orientation of the animal.

19. The system as claimed in claim 16, wherein the food supply is operable based on the positional information of the animal.

20. The system as claimed in claim 11, further comprising:
stimulating means configured to provide a second stimulus different from the first stimulus to the animal; and
monitoring means configured to monitor the activity of the animal in response to a combination of the first and second stimulus.

21. The system as claimed in claim 20, wherein the second stimulus comprises one selected from a group consisting of a light, a sound, an electric current, an odour and a temperature change.
